(19) United States
(12) Patent Application Publication
(10) Pub. No.: US 2026/0165766 A1
(43) Pub. Date: Jun. 18, 2026

(54) 360 DEGREE TURBINE DIFFUSER FOR CRYOGENIC ABLATION SYSTEMS

(71) Applicant: PENTAX OF AMERICA, INC., Montvale, NJ (US)

(72) Inventors: Steven DANIEL, Pioneer, CA (US); Parth DESAI, San Jose, CA (US)

(73) Assignee: PENTAX OF AMERICA, INC., Montvale, NJ (US)

(21) Appl. No.: 18/986,613

(22) Filed: Dec. 18, 2024

(51) Int. Cl. A61B 18/02; A61B 17/00; A61B 18/00; A61M 25/10

(52) U.S. Cl. CPC A61B 18/0218; A61B 2017/00973; A61B 2018/00208; A61B 2018/0022; A61B 2018/00577; A61B 2018/0091; A61B 2018/0212; A61M 2025/105

(57) ABSTRACT

A rotational turbine diffuser for cryogenic ablation includes a non-rotating discharge core having an external portion at the distal end and an internal portion that is fluidly coupled to a delivery tube by a lumen at the proximal end to receive refrigerant, and to distribute refrigerant via internal pathways to, a rotating discharge disk having a plurality of cavities for conducting refrigerant to a plurality of orifices in the rotating discharge disk, that dispense refrigerant radially outward in a stream, causing the discharge disk to rotate about the internal portion of the discharge core, thereby broadcasting the refrigerant onto an inner surface of the balloon in a 360 degree discharge pattern, a discharge cover covering the cavities forming viaways for the refrigerant, and a proximal retainer fixedly mounted to the discharge core on the proximal end securing the discharge disk and discharge cover.

### CROSS-REFERENCE TO OTHER APPLICATIONS

This application is related to the following: (i) U.S. patent application Ser. No. 14/530,288, titled “Cryogenic Balloon Ablation System”, filed 31 Oct. 2014, now U.S. Pat. No. 9,050,073, issued 9 Jun. 2015, attorney docket WILL 1004-2; (ii) U.S. patent application Ser. No. 14/714,101, titled “Cryogenic Balloon Ablation System”, filed 15 May 2015, now U.S. Pat. No. 9,414,878, issued 16 Aug. 2016, attorney docket WILL 1006-1; and (iii) U.S. patent application Ser. No. 15/593,790, titled “Cryogenic Ablation System with Rotatable and Translatable Catheter”, filed 12 May 2017, now U.S. Pat. No. 10,251,693, issued 9 Apr. 2019, attorney docket WILL 1007-2. The disclosures of each are incorporated by reference.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves may also correspond to implementations of the claimed technology.

Throughout the human body there are lumens, such as the esophagus and colon, which may have components which may become metaplastic or neoplastic. Often, it is desirable to remove or destroy these unwanted tissues. One of these cases where tissue removal and/or ablation are desirable is Barrett's Esophagus, which is a pre-cancerous condition of the esophagus typically often associated with gastric reflux disease (GERD). Although GERD can be medically controlled, Barrett's Esophagus does not spontaneous resolve once the GERD has abated. However, it has been shown that if Barrett's Esophagus is ablated, the normal esophagus lining can be restored and therefore lower the risk of developing esophageal cancer. Ablation therapy using both high and low temperature is well known. High temperature tissue ablation typically consists of applying various forms of electricity, highly focused ultrasound, hot fluid, steam, or lasers to raise the tissue temperature to a lethal level. In contrast, cryo-ablation lowers the tissue temperature to a point of cell necrosis. The cryo-ablation temperatures typically are generated with the application of fluids such as liquid nitrogen, nitrous oxide, or the like. The cryogenic fluid is applied on or next to the target tissue. As the fluid changes phase from a liquid to a gas, energy is absorbed from the targeted tissue or tissue area thereby lowering the tissue temperature to a lethal level.

Conventional ablation devices suffer from several shortcomings. High temperature ablations tend to char the target tissue. This process creates significant post procedure pain for the patient. The dehydration and charring of tissue also creates a non-uniform energy distribution since the char creation is unpredictable and the char itself acts as an electrical and thermal insulator. Once charred, the tissue will force a redistribution of energy creating potential overdosing of tissue.

A variety of low-temperature techniques have been evaluated for ablation. These techniques include cryogenic ablation via a direct spray of liquid nitrogen (cryogen). Challenges arise in treating these types of lesions with cryogenic ablation relate to delivery of sufficient refrigerant for ablation over a large lesion area, consistency in the evenness of application over the lesion area, performing an appropriate number of passes needed to provide effective coverage, and/or the time needed to complete treatment(s).

### BRIEF SUMMARY

A simplified summary is provided herein to help enable a basic or general understanding of various aspects of exemplary, non-limiting implementations that follow in the more detailed description and the accompanying drawings. This summary is not intended, however, as an extensive or exhaustive overview. Instead, the sole purpose of this summary is to present some concepts related to some exemplary non-limiting implementations in a simplified form as a prelude to the more detailed description of the various implementations that follow. Reference numerals are sometimes used to refer to elements of disclosed examples and not in a limiting sense.

A rotational turbine diffuser includes a non-rotatable discharge core 404 having an external portion 404a at the distal end and an internal portion 404b that is fluidly coupled to a delivery tube 30 by a lumen 43 at the proximal end to (i) receive refrigerant, and (ii) distribute the refrigerant via internal pathways 41; a rotatable assembly 400 to receive the refrigerant and having a first plurality of cavities 49 for conducting the refrigerant to a plurality of orifices 40 in the rotatable assembly 400 to dispense the refrigerant, causing the rotatable assembly 400 to rotate about the internal portion 404b of the non-rotatable discharge core 404, thereby radially diffusing the refrigerant outward in a 360 degree discharge pattern and a retainer 401 (e.g., retainer means) to secure the rotatable assembly 400 to the non-rotatable discharge core 404 and to permit the rotatable assembly 400 to rotate freely about the internal portion 404b. In some implementations, rotatable assembly 400 can comprise a discharge disk 403 tightly coupled with a discharge cover 402 by means of adhesive, or other bonding means. Alternative rotational assembly configurations may be implemented using fewer or greater number of subcomponents and using technologies such as 3D printing, casting, molding or the like. In various implementations, orifices 40 can be space approximately 180, approximately 120, approximately 90 degrees apart circumferentially about the rotatable assembly 400.

Another rotational turbine diffuser configuration includes a non-rotatable discharge core 404 having an external portion 404a at the distal end and an internal portion 404b that is fluidly coupled to a delivery tube 30 by a lumen 43 at the proximal end to (i) receive refrigerant, and (ii) distribute the refrigerant via internal pathways 41; a rotatable discharge disk 403 to receive the refrigerant and having a first plurality of cavities 49 on a first side and a discharge cover 402 covering the first plurality of cavities 49 forming viaways for conducting the refrigerant to a plurality of orifices 40 in the rotatable discharge disk 403 to dispense the refrigerant, causing at least the rotatable discharge disk 403 to rotate about the internal portion 404b of the non-rotatable discharge core 404, thereby radially diffusing the refrigerant outward in a 360 degree discharge pattern, and a retainer 401 fixedly mounted to the non-rotatable discharge core 404, securing the discharge disk 403 to the non-rotatable discharge core 404 and permitting the rotatable discharge disk 403 to rotate freely about the internal portion 404b. In various implementations, orifices 40 can be space approximately 180, approximately 120, approximately 90 degrees apart circumferentially about the rotatable discharge disk 403.

An ablation assembly 10 includes a controller assembly 13 and a cryogenic ablation catheter 12. The controller assembly 13 comprises a handle assembly 14, a controller 50, and a user control assembly 15 coupled to the controller. The handle assembly comprises a connector receptacle 99. The cryogenic ablation catheter 12 comprises a catheter shaft 16, a connector 22, an expandable and collapsible balloon 24, and a delivery tube assembly. The catheter shaft 16 has proximal and distal ends and a catheter shaft lumen extending between the proximal and distal ends. The connector 22 is at the proximal end of the catheter shaft and is selectively connected to a first connector element of the handle assembly. The connector comprises a connector body 23 and a plug 38, also called second connector element 38. The expandable and collapsible balloon 24 is mounted to the distal end of the catheter shaft, the balloon having an inner surface defining a balloon interior. A delivery tube assembly comprises a delivery tube 30 and a diffuser 36. The delivery tube 30 is housed within the catheter shaft for axial and rotational movement relative to the catheter shaft. The delivery tube has a proximal end connected to the plug. The diffuser 36 is within the balloon and is a rotational turbine diffuser that comprises (i) a non-rotating discharge core 404 having an external portion 404a at the distal end and an internal portion 404b that is fluidly coupled to the delivery tube by a lumen 43 at the proximal end, to receive refrigerant and to distribute refrigerant via internal pathways 41a rotatable assembly 400 to receive the refrigerant and having a first plurality of cavities 49 for conducting the refrigerant to a plurality of orifices 40 in the rotatable assembly 400 to dispense the refrigerant, causing the rotatable assembly 400 to rotate about the internal portion 404b of the non-rotatable discharge core 404, thereby radially diffusing the refrigerant outward in a 360 degree discharge pattern; and a retainer 401 to secure the rotatable assembly 400 to the non-rotatable discharge core 404 and to permit the rotatable assembly 400 to rotate freely about the internal portion 404b. The handle assembly comprises a handle assembly body 90, the controller connector 100, a traveler 110, a refrigerant fluid source 96, the linear driver 104, 108, 106, and a rotary motion driver 120, 124, 122. The controller connector 100 is mounted to the handle assembly body and defines the first connector element, the connector body being securable to the controller connector. The traveler 110 is movably mounted to the handle assembly body for movement along an axis towards and away from the controller connector, the plug being securable to the traveler for axial movement therewith. The refrigerant fluid source 96 is selectively fluidly coupled to a delivery line 118 by the refrigerant controller 130, 128. The delivery line has a distal end connected to the traveler, whereby the refrigerant delivery source can be fluidly coupled to the delivery tube at the plug/second connector element. The linear driver 104, 108, 106 is operably coupled to the traveler for moving the traveler along the axis. The rotary motion driver 120, 124, 122 is operably coupled to the plug for selective rotation of the plug and the proximal end of the delivery tube therewith about the axis. The user control assembly is operably coupled to the refrigerant controller, the linear driver and the rotary motion driver. The user control assembly includes user inputs permitting the user to actuate the refrigerant controller, the linear driver and the rotary motion driver. Whereby the user can control the rotation and translation of the diffuser within the balloon to direct refrigerant outwardly in a desired pattern towards the inner surface of the balloon according to, for example, the size and location of the treatment site.

Examples of the ablation assembly can include one or more the following. The user control assembly 15, also called a foot pedal assembly 15, spaced apart from the handle assembly 14 and connected to the handle assembly by a line 17, the foot pedal assembly comprising foot actuated input devices. The foot pedal assembly 15 can comprise a left movement foot pedal 140, a right movement foot pedal 142 and movement mode button 144 by which the user can change the mode of operation of the left and right movement foot pedals to actuate either the linear driver or the rotary motion driver. The foot pedal assembly 15 can also include a refrigerant delivery foot pedal 132 by which a user can actuate the refrigerant controller to supply refrigerant to the balloon 24 and a balloon deflation button 134.

Examples of the ablation assembly can also include one or more the following. The traveler can be positionable along the axis at a first, eject position, a second, load position, and at a range of third, operational positions, the first, eject position being closest to the controller connector, the second, load position being between the first, eject positions and the third, operational positions. The ablation assembly can further comprise: means for automatically securing the connector body 23 to the controller connector 100 and the plug 38, also called the second connector element 38, to the traveler 110 when the connector is inserted into the first connector element and the traveler is at the second, load position; and means for automatically releasing the connector body from the controller connector and the plug from the traveler when the traveler is at the first, eject position to permit removal of the connector from the handle assembly body.

Examples of the ablation assembly can also include one or more the following. The refrigerant fluid source 96 can include a removable and replaceable refrigerant cartridge having a tip 178 through which refrigerant can pass to the delivery line 118 via the refrigerant controller 130, 128 when the refrigerant fluid source is at an operational position. The handle assembly body 90 can include a refrigerant venting chamber 180 and a pathway 182, 184, 186 fluidly connecting the interior of the refrigerant venting chamber to a region adjacent to the tip of the refrigerant cartridge when the refrigerant cartridge has been displaced from the operational position during removal of the refrigerant cartridge from the handle assembly body 90. Whereby residual liquid refrigerant from the refrigerant cartridge can flow into the refrigerant venting chamber for transformation into a refrigerant gas, the refrigerant venting chamber having an exit port 192 to permit the refrigerant gas to exit the refrigerant venting chamber. In some examples the exit port 192 opens into the handle assembly body 90, the handle assembly body having a plurality of exhaust ports 133, 135 opening into the ambient atmosphere.

In some examples the ablation assembly can include one or more the following. The traveler can be positionable along the axis at a first, eject position, a second, load position, in the range of third, operational positions, the first, eject position being closest to the controller connector, the second, load position being between the first, eject positions and the third, operational positions. The rotary motion driver can include a rotation motor 120, a drive gear 126 and gear teeth 85. Rotation motor 120 can be drivingly connected to a non-cylindrical rotation shaft 122. The drive gear 126 can be mounted to traveler 110 for axial movement with the traveler, the drive gear also slideably mounted to the rotation shaft. Whereby rotation of the rotation shaft causes the drive gear to rotate and axial movement of the traveler causes the drive gear to slide along the rotation shaft. The gear teeth 85 can be formed on the plug 38 and rotatably coupled to the drive gear when the traveler is in either the second, load position or the third, operational position, so that rotation of the drive gear causes the plug 38 to rotate. The linear driver can include a linear drive motor 104 connected to a threaded shaft 106 by a threaded shaft coupler 108, the threaded shaft threadably engaging the traveler 110 so that rotation of the threaded shaft causes the traveler to move axially.

In some additional examples, the ablation assembly can include one or more the following. The connector 22 can include an RFID device 198 containing information relating to the cryogenic ablation catheter 12, and handle assembly can include an RFID reader 200 used to obtain information from the RFID device. The RFID reader 200 can be mounted to the controller connector 100, the connector body 23 being made of PEEK (polyetheretherketone) to enhance the communication between the RFID reader and the RFID device 198. The controller assembly can include a controller 50, and the user control assembly can be operably coupled to the refrigerant controller, the linear driver, and the rotary motion driver through the controller. A pressure detecting lumen 32 can extend along the catheter shaft 16 fluidly coupling the balloon interior and the connector body 23, and the controller 50 can be configured to use input received from a pressure transducer 143 operably coupled to the pressure detecting lumen 32 through the connector body 23 to detect a pressure within the balloon 24. The controller 50 can be configured to monitor the pressure and temperature of the refrigerant fluid source 96, whereby the status of the refrigerant can be monitored. The first connector element can include a connector receptacle and the second connector element can include a plug.

An example of a second ablation assembly includes a handle assembly 14, a catheter 12, and a connector locking assembly. The catheter 12 includes a catheter shaft 16 having distal and proximal ends, a balloon 24 at the distal end of the catheter shaft, a connector 22 at the proximal end of the catheter shaft, and a delivery tube 30 extending between the balloon and the proximal end of the catheter shaft. The connector includes a connector body 23 secured to the proximal end of the catheter shaft and a plug 38 secured to the delivery tube, the plug and delivery tube therewith movable axially and rotationally relative to the catheter shaft. The handle includes an open portion for receipt of the plug and at least a portion of the connector body. The connector locking assembly includes connector body and plug clocking slots. The connector body locking slot 74 is formed in and circumscribes the connector body 23. The plug locking slot 84 is formed in and circumscribes the plug 38. The connector locking assembly also includes connector body and plug locking elements. The connector body locking element 160 is mounted to the handle and positioned to engage the connector body locking slot 74 when the connector 22 is in a load state. The plug locking element 152, 154 is mounted to the handle and positioned to simultaneously engage the plug locking slot 84 when the connector is in the load state, thereby simultaneously automatically connecting the plug 38 and the connector body 23 to the handle to place the connector in a load state prior to use. The connector body locking element 160 it is mounted to the handle and positioned to disengage from the body locking slot when the connector is being placed in an eject state. The plug locking element 152, 154 is mounted to the handle and positioned to disengage from the body locking slot when the connector is in the eject state, thereby automatically releasing the connector body 23 and thereafter the plug 38 from the handle to permit the connector to be removed from the handle.

Some examples of the second ablation assembly can include one or more the following. The connector body locking element can include a first spring 160 and the plug locking element can include a second spring 152, 154. The connector body 23 can include a tapered surface 162 engageable by the first spring 160 when the connector is placed into the load state, and the plug 38 can include a tapered surface 156 engageable by the second spring 152, 154 when the connector is placed into the load state. The connector locking assembly can include a ramp 170 engageable by the first spring 160 when the connector is placed into the eject state; and can include the connector locking assembly comprises a ramp 174 engageable by the second spring 152, 154 when the connector is placed into the load state.

An example of a third ablation assembly includes a handle assembly 14, a catheter 12 and a connector locking assembly. The catheter 12 includes: a catheter shaft 16 having distal and proximal ends, a balloon 24 at the distal end of the catheter shaft, a connector 22 at the proximal end of the catheter shaft, and a delivery tube 30 extending between the balloon and the proximal end of the catheter shaft. The connector includes a connector body 23 secured to the proximal end of the catheter shaft and a plug 38 secured to the delivery tube, the plug and delivery tube therewith movable axially and rotationally relative to the catheter shaft. The handle includes an open portion for receipt of the plug and at least a portion of the connector body. The connector locking assembly includes: means for simultaneously automatically connecting the plug 38 (84, 152, 154, 156, 158) and the connector body 23 (160, 174, 162) to the handle to place the connector in a load state prior to use, and means for automatically releasing the connector body (74, 110, 160, 170) and thereafter the plug (84, 110, 152, 154, 174) from the handle to place the connector in an eject state to permit the connector to be removed from the handle.

An example of a handle assembly for use with a cryogenic ablation assembly 10 includes a handle body 90, having anterior, and a refrigerant fluid source 96 within the interior. The refrigerant fluid source 96 includes a refrigerant discharge portion 178. The refrigerant fluid source includes a removable and replaceable refrigerant cartridge 96 from which refrigerant can pass for use by the cryogenic ablation assembly 10 when the refrigerant fluid source is at an operational position. The handle body 90 includes a refrigerant venting chamber 180 and a pathway 182, 184, 186 fluidly connecting the interior of the refrigerant venting chamber to a region adjacent to the refrigerant discharge portion of the refrigerant cartridge when the refrigerant cartridge has been displaced from the operational position during removal of the refrigerant cartridge from the handle body 90. The handle body includes an exhaust port 133, 135 opening into the ambient atmosphere. Residual liquid refrigerant from the refrigerant cartridge can flow into the refrigerant venting chamber for transformation into a refrigerant gas. The refrigerant venting chamber has an exit port 192 to permit the refrigerant gas to exit the refrigerant venting chamber and into a region external of the refrigerant venting chamber 180 and within the handle body 90, for passage through the exhaust port to the ambient atmosphere.

Examples of the handle assembly can include one or more the following. The refrigerant venting chamber 180 can be filled with a material with an entrance path 188 and an exit path 190 formed in the material, the entrance path connected to the pathway and the exit path 190 terminating at the exit port 192. Whereby under a reduced pressure within the refrigerant venting chamber 180, the liquid refrigerant can be absorbed by the foam material and transformed into a gas for collection within the exit path 190, passage through exit port 192 into said region external of the refrigerant venting chamber 180 and within the handle body 90. A thermal insulation material 194 can be used between the refrigerant venting chamber 180 and the handle body 90, and spacers 196 can be used between the thermal insulation material 194 and the handle body 90.

An example of a catheter identification structure, for a cryogenic ablation assembly 10 of the type including a handle assembly 14 and a catheter assembly, the catheter 12 including a catheter shaft 16 having distal and proximal ends, a connector 22 at the proximal end of the catheter shaft, and a connector 22, the handle assembly 14 including an open portion for receipt of at least a portion of the connector, the catheter identification structure including an RFID device 198 and an RFID reader 200. The RFID device 198 is carried by the connector 22 and contains information relating to the catheter 12. The RFID reader 200 is carried by the handle assembly and is used to obtain information from the RFID device.

In some examples of the catheter identification structure, the connector 22 includes a connector body 23 made of PEEK to enhance the communication between the RFID reader 200 and the RFID device 198.

Implementations can provide features and advantages such as without limitation, producing 360-degree cylindrical ablation in a single treatment cycle, greater consistency and uniformity in ablations with a continuous rotating/translating discharge of cryogen; removal of over treatment and/or under treatment inherent with conventional multiple discharge orifices; reducing or eliminating a need for overlapping smaller ablations thereby eliminating over dosing and/or under dosing of tissue; reducing sensitivity to poor alignment and poor placement of the diffuser; reducing procedure and anesthesia time by eliminating one or more of multiple placements, the time to place/target the diffuser, and the time for making multiple ablations; greater ease of use under direct visualization; reducing or eliminating a need to use fluoroscopy; providing variable ablation diameter, length of ablation, and dosing to better fit a patient's needs; and/or reducing or eliminating a need for thermal blocking or absorption measures such as submucosal injections in order to protect deeper or surrounding tissue. These and other features, aspects and advantages of the technology disclosed can be seen on review the drawings, the detailed description, and the claims which follow.

### BRIEF DESCRIPTION OF THE DRAWINGS

The included drawings are for illustrative purposes and serve only to provide examples of possible structures and process operations for one or more implementations of this disclosure. These drawings in no way limit any changes in form and detail that may be made by one skilled in the art without departing from the spirit and scope of this disclosure. A more complete understanding of the subject matter may be derived by referring to the detailed description and claims when considered in conjunction with the following figures, wherein like reference numbers refer to similar elements throughout the figures.

FIG. 1 is a simplified schematic overall view of an example of an ablation system including a cryogenic balloon ablation assembly and an endoscope suitable for use with a turbine diffuser.

FIG. 2A illustrates an ablation catheter with a deflated balloon in tension.

FIG. 2B illustrates an ablation catheter with an inflated balloon with the diffuser located at a distal region of the balloon.

FIG. 2C illustrates an ablation catheter with an inflated balloon with the diffuser located in a proximal region of the balloon.

FIG. 3A is a cross-sectional view taken along line 3A-3A of FIG. 2A.

FIG. 3B is a cross-sectional view taken along line 3B-3B of FIG. 2A.

FIG. 3C is a cross-sectional view taken along line 3C-3C of FIG. 2A.

FIG. 4A illustrates an isometric view of a 360 degree rotational turbine diffuser.

FIG. 4B is a cross-sectional view taken along line 4B-4B of FIG. 4A.

FIG. 4C is a cross sectional view of the 360 degree rotational turbine diffuser taken along line 4C-4C of FIG. 4B showing flow paths.

FIG. 4D shows orifices disposed on both sides of a discharge disk of a rotational turbine diffuser.

FIG. 4E shows tubes inserted in viaways to reduce orifice size a rotational diffuser.

FIG. 4F shows a diffuser having a helical cut element that is free to rotate with passage of the cryogenic fluid.

FIG. 4G shows a 360-degree orifice with a proximal helical cryogen flow path.

FIG. 4H shows multiple discharge orifices oriented in a 360-degree arrangement at the distal end of cryogen delivery tube in a non-rotational diffuser design.

FIG. 4I shows diffuser having a full circumferential 360-degree orifice tip.

FIG. 5A illustrates an external view of an example non-rotational diffuser.

FIG. 5B is a cross section of the diffuser of FIG. 5A.

FIG. 5C is a cross section of the diffuser of FIG. 5A showing flow paths.

FIG. 6A is a detailed view of the balloon of FIG. 2A.

FIG. 6B is an isometric cross section view of the balloon of FIG. 2B.

FIG. 6C is a cross section view of the balloon of FIG. 2B.

FIG. 6D is an isometric cross section view of the balloon of FIG. 2C.

FIG. 7A illustrates an enlarged cross section view of the connector of FIG. 2A.

FIG. 7B illustrates the connector of FIG. 7A with flow lines.

FIG. 7C illustrates an enlarged cross section view of the connector of FIG. 2B omitting the flow paths and showing the plug pulled distally in short distance.

FIG. 7D is an enlarged isometric view of the plug of FIG. 7C.

FIG. 8 is a partially exploded isometric view of portions of the ablation assembly of FIG. 1.

FIG. 8A is a right-side cross section view of the handle assembly.

FIG. 8B is a left side cross section view of the handle assembly.

FIGS. 8C and 8D show the relationship of the catheter plug locking wire relative to the plug locking slot of the plug when the ablation assembly is in the catheter connector disconnect state of FIGS. 13 and 13A.

FIG. 8E is a simplified schematic cross section the connector body locking wire biased away from the body locking slot of the connector body.

FIG. 9 is a cross-sectional view of the handle assembly in the controller load position.

FIG. 9A is an enlarged view of a portion of the structure of FIG. 9.

FIGS. 9B and 9C show the catheter plug locking wire engaging the plug locking slot.

FIG. 9D shows the structure of FIG. 8E but with the connector body locking wire engaging the body locking slot of the catheter body.

FIGS. 9E and 9F illustrate how the pressure within the balloon is communicated to a pressure transducer in the controller connector.

FIG. 10 is a partial cross-sectional view of the traveler of FIG. 8A showing the idler gear connecting the drive gear and the purity of the plug.

FIG. 11 shows isometric views of portions of the handle assembly at the start of treatment.

FIG. 12 shows the structure of FIG. 11 at the end of treatment.

FIG. 13 shows the structure of FIG. 12 in a catheter connector disconnect state.

FIG. 13A is a left side partial cross-sectional view of the handle assembly in the catheter-disconnect state of FIG. 13.

FIGS. 13B and 13C are top views of the structure of FIG. 13.

FIGS. 14, 14A, 14B and 14C illustrates how excess liquid refrigerant is directed into a refrigerant venting chamber: removal of the refrigerant canister from the handle assembly.

FIG. 15 illustrates how the handgrip portion of the handle assembly body is thermally insulated from the refrigerant venting chamber.

FIGS. 16 and 17 illustrate a linear positioner used to ensure the proper linear position of the traveler relative to the controller connector.

FIG. 18 is a simplified controller hardware architecture design chart.

### DESCRIPTION

The following description will typically be with reference to specific structural embodiments and methods. It is to be understood that there is no intention to be limited to the specifically disclosed embodiments and methods but that other features, elements, methods and embodiments may be used for implementations of this disclosure. Preferred embodiments are described to illustrate the technology disclosed, not to limit its scope, which is defined by the claims. Those of ordinary skill in the art will recognize a variety of equivalent variations on the description that follows. Unless otherwise stated, in this application specified relationships, such as parallel to, aligned with, or in the same plane as, mean that the specified relationships are within limitations of manufacturing processes and within manufacturing variations. When components are described as being coupled, connected, being in contact or contacting one another, they need not be physically directly touching one another unless specifically described as such. Like elements in various embodiments are commonly referred to with like reference numerals.

Existing cryo-ablation devices are limited in the size and area which they can effectively treat per application. Not being able to treat the full target tissue with a single application forces the user to make multiple applications to achieve the required result. Inevitably the practical limitation of accurately applying multiple treatments results in areas of under or overlapping treatments. Overlapping treatments usually result in excessive dosing to the targeted tissue. Adverse effects of overtreatment can include stricturing of the tissue, perforations, and the like. Conversely undertreatment of tissue caused by gaps between treatments can also result from targeting limitations of the current devices during multiple applications. This will typically result in recurrences and/or a spreading of the condition, such as in the case with cancer.

Another limitation of the current cryo-technology is the overall nonuniformity of the treatment. This inconsistency can be created, for example, by overlapping areas from the discharge of multiple fixed orifices or diffusers. The over or underlapping discharge from a single or group of fixed diffusers results in areas of higher and lower dosing, over or under treatment. This inconstancy again causes the tissue to be over or under treated within the ablation. As described previously, this can have extremely severe or life threating consequences.

Controlling the amount of energy delivered to the target tissue is another limitation of the current diffuser approaches. Some current devices and methods do not have the ability to consistently deliver the requisite dose; for example, one conventional approach includes delivering an excessive dose to the target, coupled with insulating the deeper or surrounding tissue. This insulation or dissipation of the delivered energy cam be used to prevent excessive energy being absorbed into the deeper tissue layers causing severe tissue damage and patient harm.

Yet another limitation of conventional approaches that include delivering multiple successive treatments is the amount of time needed to make multiple successive treatments. The duration of procedures and the amount of anesthesia time has both a procedural cost impact as well as making patient recovery more difficult. Prolonged procedures with extended anesthesia exposure can be life threatening for some patients. The prospect of prolonged procedures can exclude these patients from the possibility of receiving a treatment they need.

Still another limitation of conventional diffuser technologies is that conventional approaches often rely on manually implemented “rotational control” by the clinician (or other operator) over the diffuser for distributing cryogen radially. This conventional approach (described in greater detail herein below) requires the user to control the rotation and axial translation of the diffuser manually with a control. This manual control, while affording certain advantages, can result in uneven distribution of the cryogen.

A still yet further limitation of conventional diffuser technologies is the lack of direct visibility during use. Some devices require extraordinary measures such as the use for fluoroscopy in order to perform the procedure. Use of fluoroscopy extends procedure times and causes radiation exposure for both the patient and caregivers which is highly undesirable.

These and other shortcomings can be overcome by 360-degree rotational turbine diffuser implementations that employ rotating and translating orifice(s) for the application of a cryogen as will now be described in further detail. Rotation of the orifice(s) is accomplished by means of cryogenic effluent escaping a pressure chamber(s), inducing the rotation. During the rotation of the orifice(s), cryogen is distributed radially 360-degrees around a central axis of the target tissue. With each orifice spraying a full 360-degrees, the overlapping and underlapping of fixed orifices is eliminated. Simultaneously while the diffuser is rotating and distributing the cryogen 360-degrees, it/they are translated axially. This creates a 360-degree cylindrical treatment area including the full circumference by the length of traverse.

With the orifice(s) or diffuser having a sufficient speed of rotation and the correct axial translation speed, the amount of cryogen, and thereby the thermal dosing, can be set and maintained. This allows for an evenly distributed cryogen at the desired amount over the entire surface of the target treatment area. In this way the need for overlapping ablations to treat larger target areas is eliminated.

This technological approach allows implementations to treat relatively larger target areas than those of conventional approaches in a single use in a few seconds without the need to reposition or retarget the treatments. As such, these technological approaches further allow implementations to achieve a shortened treatment cycle as compared with conventional approaches. Shorter treatments allow for reducing the total (time) duration of anesthesia which benefits patients under treatment and reduces the overall procedure cost. The amount of cryogen applied to the target tissue can be more precisely applied with the current invention. This improved precision eliminates the need for thermal blocking techniques to prevent deeper tissue destruction. The technology is also well suited to implementations allowing for direct visualization with a flexible scope to facilitate positioning; thereby eliminating the need for using fluoroscopy or the like.

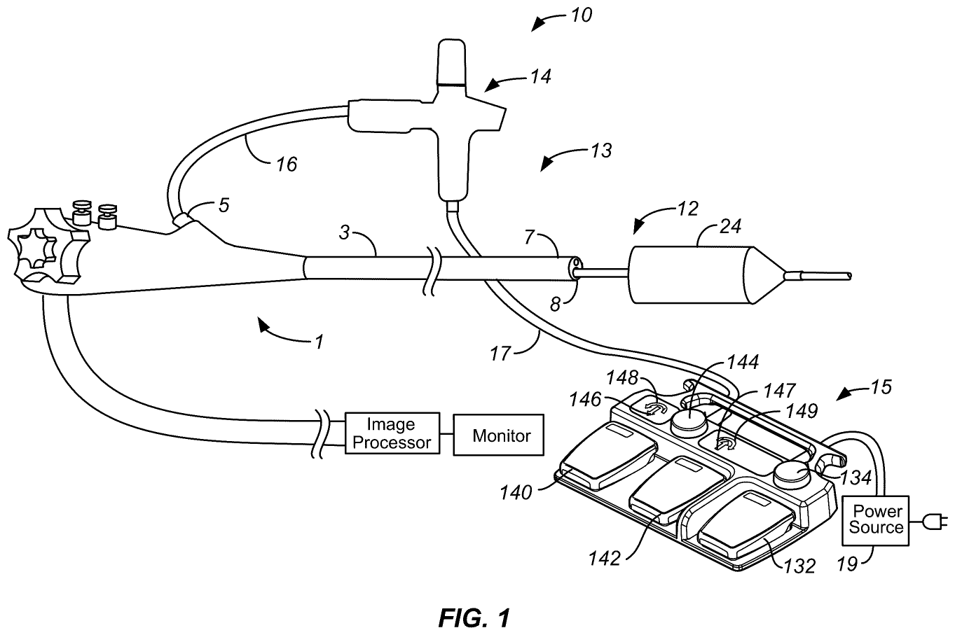

An embodiment of an ablation system suitable for use with 360-degree rotational turbine diffuser to provide improved refrigerant delivery area is shown in FIG. 1 and comprises an endoscope 1 and a cryogenic balloon ablation assembly 10. The endoscope 1 may be conventional and include an endoscopic tube 3 having proximal and distal ends 5, 7 defining a channel 8 extending between the proximal and distal ends.

In embodiments, an ablation assembly 10 comprises a cryogenic ablation catheter 12 and a controller assembly 13. Controller assembly 13 includes a handle assembly 14 and a foot pedal assembly 15 connected to handle assembly 14 by a power and control line 17. Power is supplied to controller assembly 13 by a power source 19 connected to foot pedal assembly 15. Ablation catheter 12 includes a catheter shaft 16 mounted to and extending from handle assembly 14.

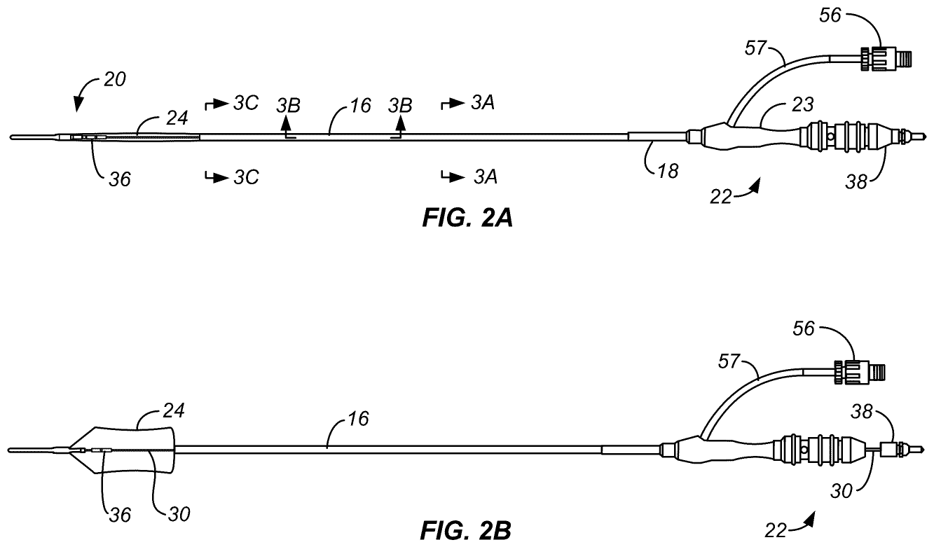

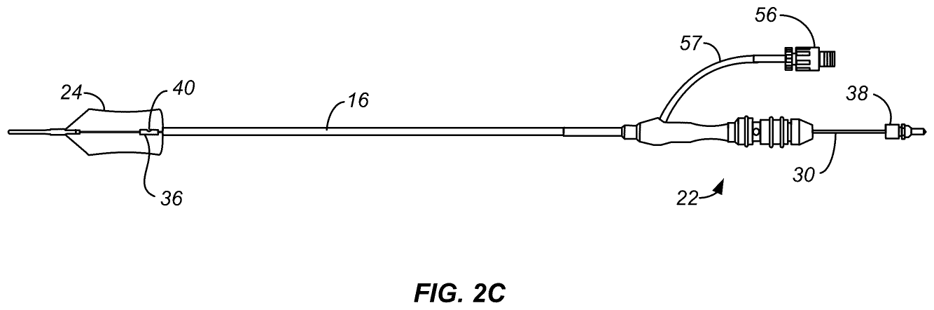

FIGS. 2A, 2B and 2C show an embodiment of a cryogenic ablation catheter in three states that will be discussed below. Catheter 12 includes a catheter shaft 16 having a proximal end 18 and a distal end 20. At the proximal end 18 is a connector 22 to be received in the handle assembly 14. Connector 22 includes a connector body 23 and a plug 38, also called the second connector element 38. At the distal end 20 is a balloon 24 that is inflatable by a refrigerant delivered from a refrigerant fluid source in the handle assembly 14 to a diffuser 36 located within the balloon. Diffuser 36 is secured to and is fluidly coupled to the distal end of a delivery tube 30. The diffuser 36 translates within the balloon through the axial movement of delivery tube 30 as is discussed in greater detail below. As illustrated in FIGS. 2B and 2C, translation of the diffuser 36 is caused by translation of a plug 38 affixed and fluidly coupled to the delivery tube 30 at the connector end of the catheter 12. As suggested by FIGS. 2B and 2C, plug 38 and delivery tube 30 and diffuser 36 therewith can be rotated to direct refrigerant in selected rotational directions and along selected rotational paths.

The catheter shaft 16 comprises a circular tube with a circular central lumen. The catheter shaft 16 may, for example, range from 120 cm (47.2 inches) to 350 cm (137.8 inches) in length and have an outer diameter ranging from, for example, 0.254 cm to 0.35052 cm (0.100 inches to 0.138 inches). The proximal end of the catheter shaft 16 is affixed to the connector 22, and the distal end is affixed to the balloon 24.

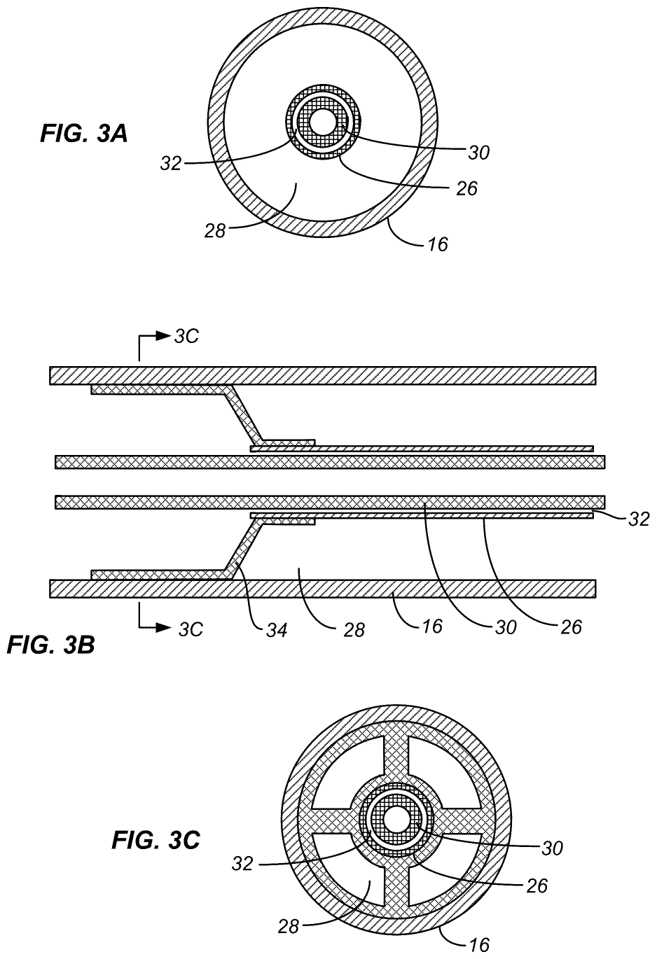

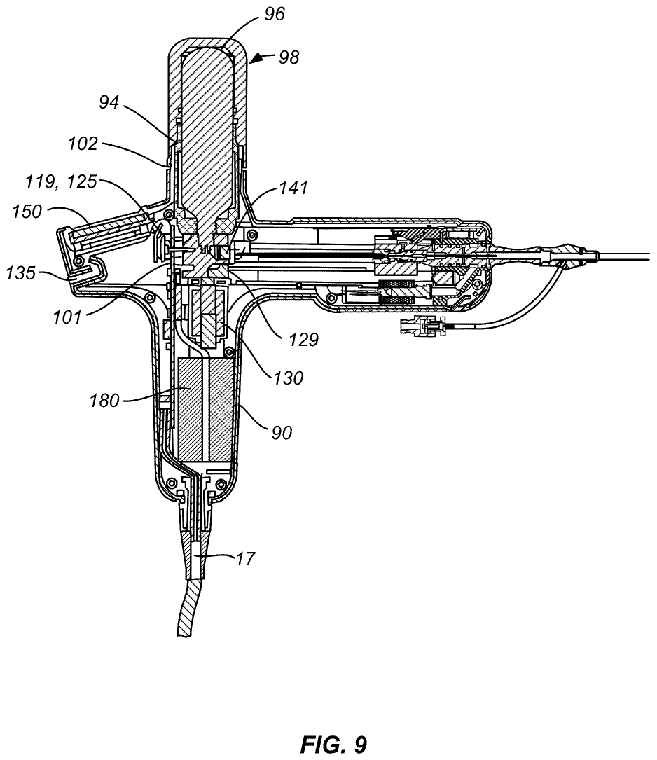

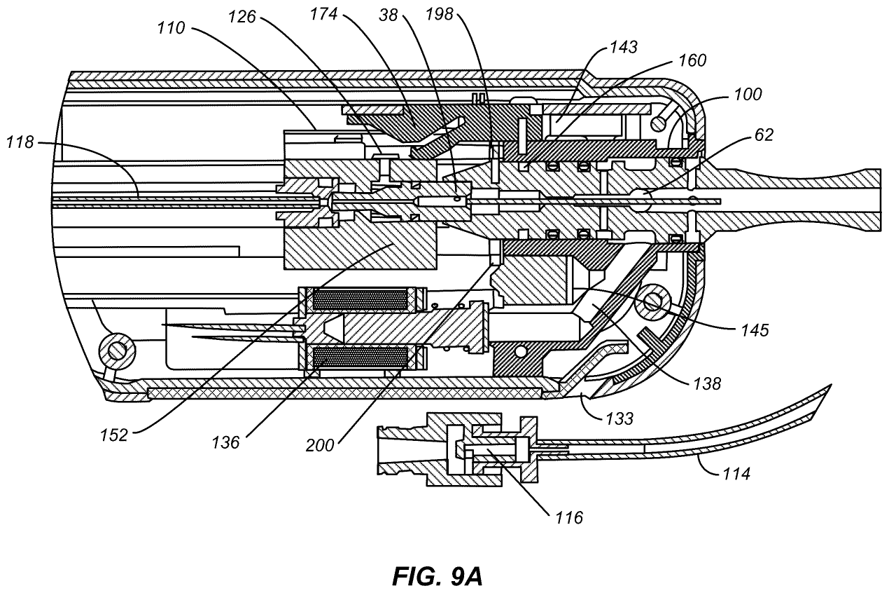

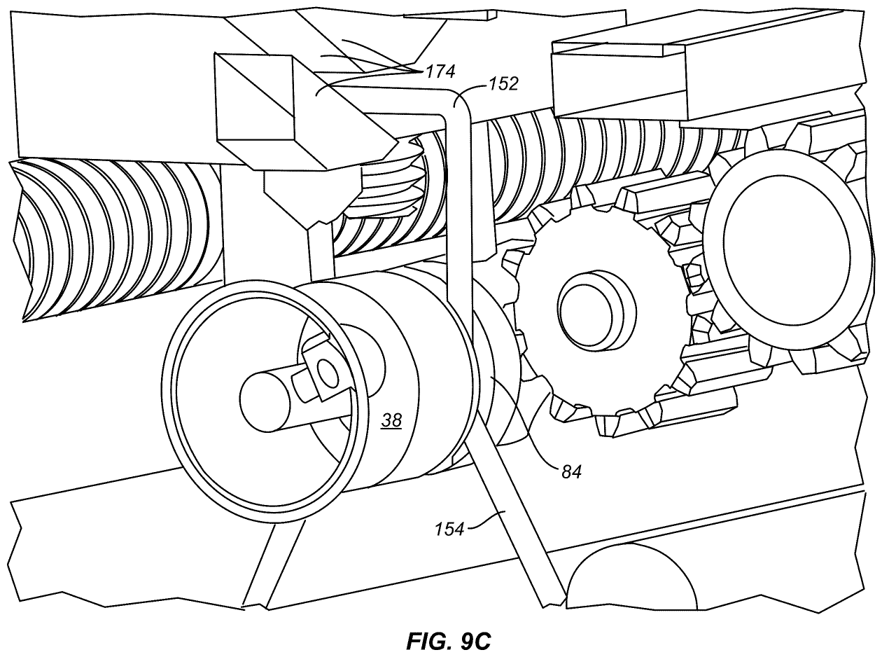

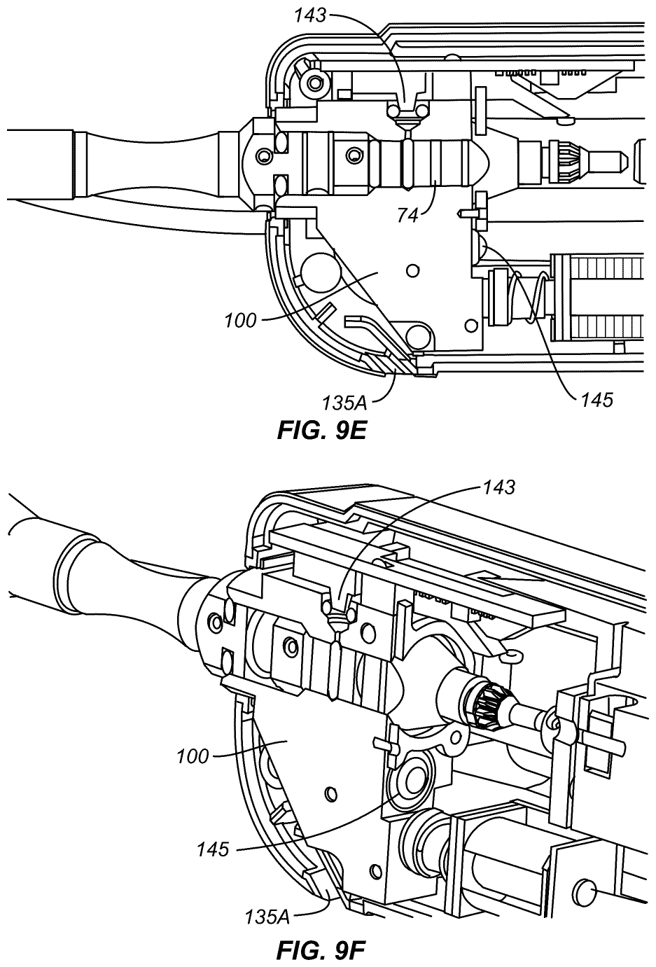

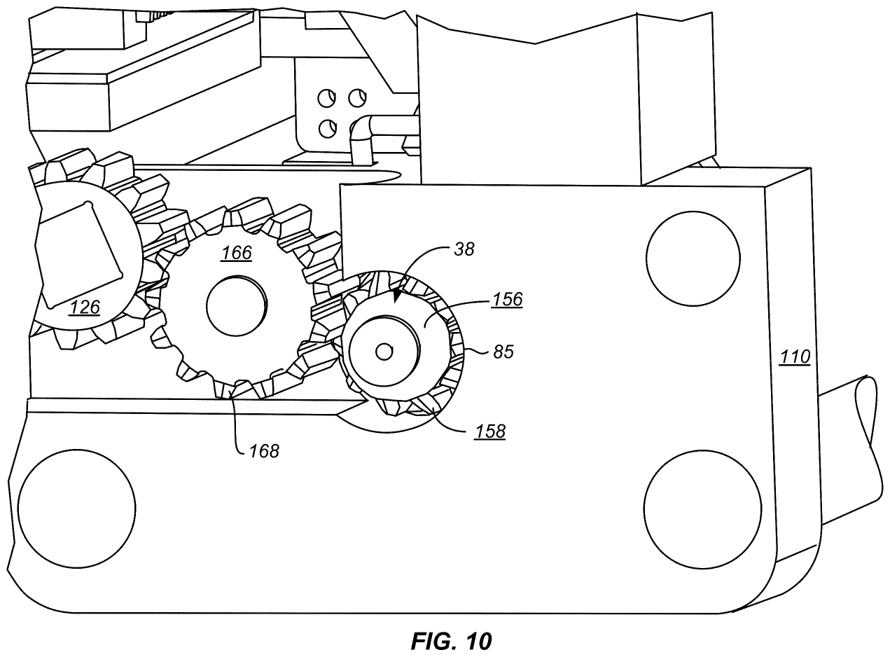

FIG. 3A shows cross-section taken along line 3A-3A of FIG. 2A. As shown, located within the catheter shaft 16 is a pressure detecting tube 26. The cavity between the inner wall of the catheter shaft and the outer wall of the pressure detecting tube defines an exhaust lumen 28 for the passage of gases from the balloon interior for discharge through the connector 22 and exhaust passageway 138 in controller connector 100, see FIG. 9A, which will be discussed in greater detail below. The pressure detecting tube 26 includes a circular central lumen containing a delivery tube 30. The cavity between the inner wall of the pressure detecting tube 26 and the outer wall of the delivery tube 30 defines a pressure detecting lumen 32 used to detect the pressure within the balloon 24.

The pressure detecting tube 26 extends from near the balloon end of the catheter shaft 16 to the connector 22. The pressure detecting tube 26 is affixed to the catheter shaft 16 near the distal end 20 of the catheter shaft 16 to be concentric to the catheter shaft lumen. The affixing means includes a bracket 34 with minimal flow obstruction of the exhaust lumen as shown in FIGS. 3B and 3C. In embodiments, the bracket 34 is positioned a distance within the end of the catheter shaft 16 so that a portion of the diffuser 36 may enter a portion of the catheter shaft 16 to allow delivery of refrigerant to portions of the balloon 24 located proximate to the distal end 20 of the catheter shaft 16.

As shown in FIG. 3A, within the pressure detecting tube 26 is the delivery tube 30. The pressure detecting tube 26 is a guide for the delivery tube 30 ensuring consistent 1:1 translation and rotation of the delivery tube within the pressure detecting tube without backlash. To facilitate this, the interior surface of pressure detecting tube 26 is a low friction surface, such as can be achieved by coating the interior surface with PTFE and the exterior surface of the delivery tube 30 with PTFE. For example, when the proximal or plug end of the delivery tube 30 is translated 4 mm (0.157 inches) and rotated 90°, then the diffuser end, and this diffuser 36, is also translated 4 mm (0.157 inches) and rotated 90°.

The delivery tube 30 extends from the plug 38 through the connector 22; through the pressure detecting tube 26, and to the diffuser 36. The delivery tube 30 is made of a strong, flexible tubing or tubing assembly. For example, the delivery tube 30 may be comprised of a tubing assembly including an outer nitinol tube, which is very elastic and does not plastically deform (e.g. kink) easily, and an inner thin-walled polyimide tube. The nitinol tubing provides structural support for the tubing assembly. The nitinol tubing provides the strength necessary to prevent buckling during axial translation of the delivery tube. Further, the nitinol tubing transmits torque well which allows for rotational movement of the delivery tube. In embodiments, the outer tube of a delivery tube assembly is a torque tube comprising stainless steel wires that undergo processes such as swaging, stretching, annealing, and then is wound around the inner tube to form a tubing assembly with good rotational and axial translation capabilities. The thin-walled polyimide inner tube is made with tight tolerances which allows for consistent flow of refrigerant through the delivery tube. The delivery tube during use may experience internal pressures of 4,137 kPa to 8,270 kPa (600 psi to 1200 psi) and may be configured to have a wall thickness to withstand internal pressures up to 10,340 kPa (1500 psi). The delivery tube 30 translates within the pressure detecting tube 26 in response to movement of the plug/second connector element 38 relative to the connector 22.

FIG. 2A shows a state of the plug 38, wherein the plug 38 abuts the connector body 23 and the diffuser 36 is located at a position toward the distal end of the balloon 24, which is shown in a deflated state. FIG. 2B shows a state of the plug 38, wherein the plug 38 is located at a first intermediate position relative to the connector 22 and the diffuser 36 is located at a position in the balloon 24, shown in an inflated state. FIG. 2C shows a state of the plug 38, wherein the plug 38 is located at a proximal position and rotated 90° which changes the orientation of nozzle port 40 90°.

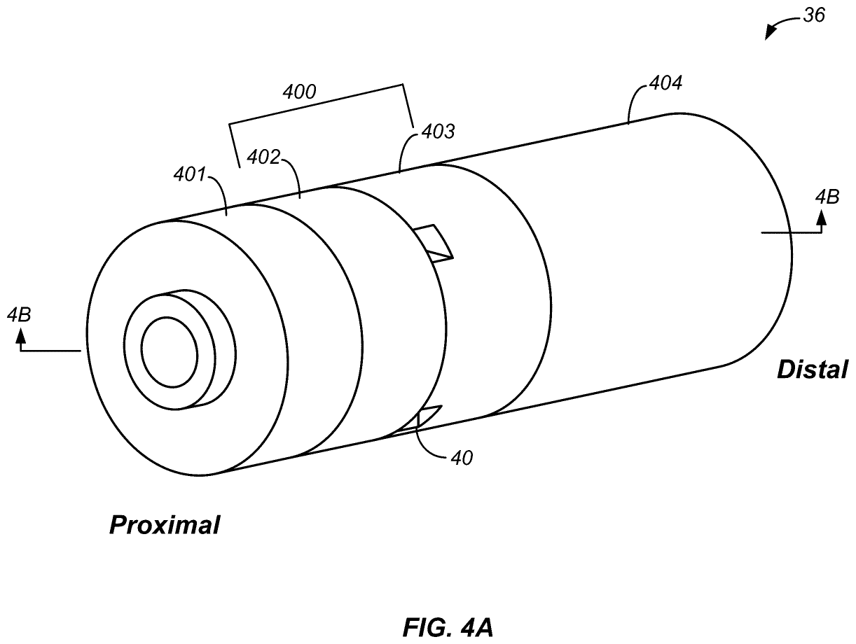

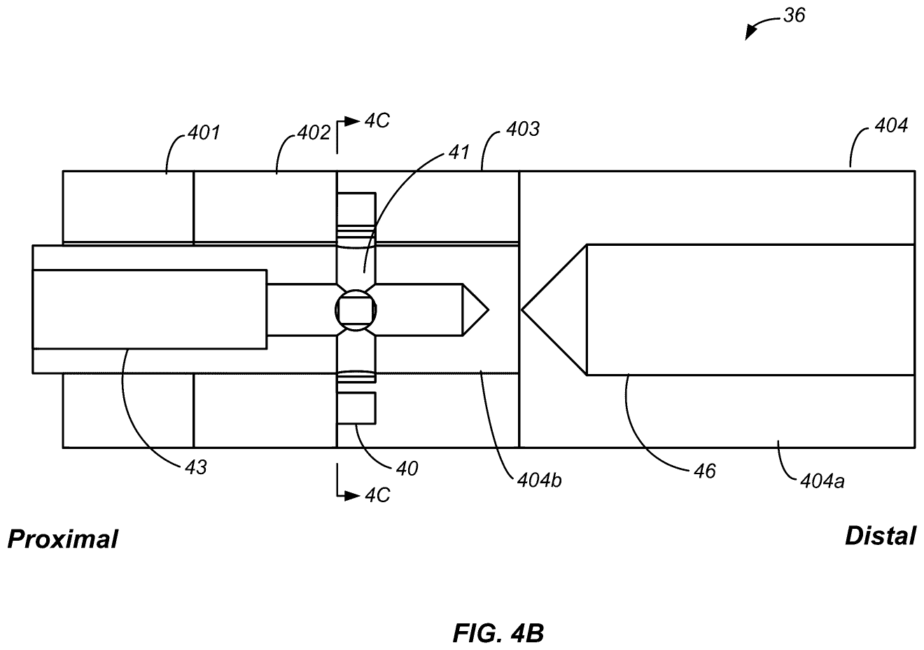

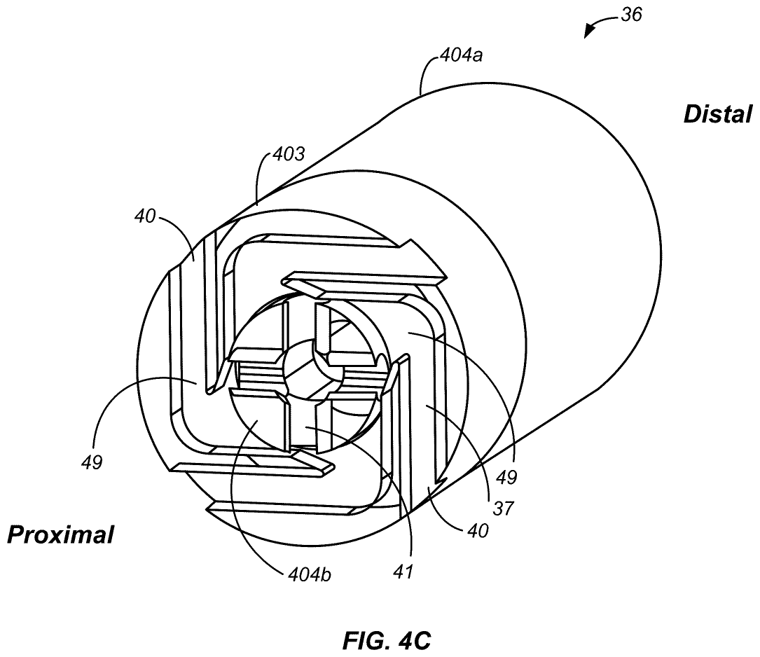

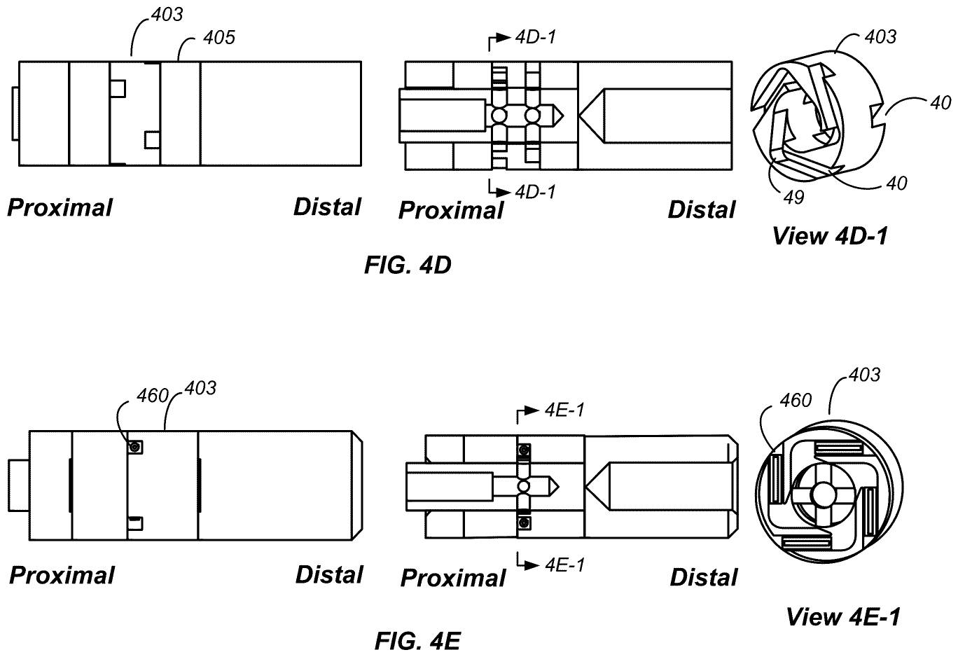

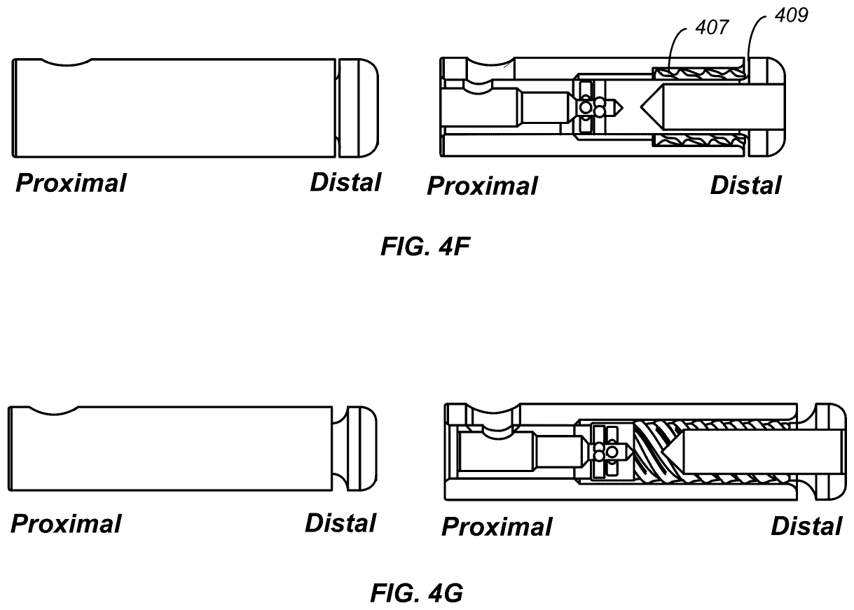

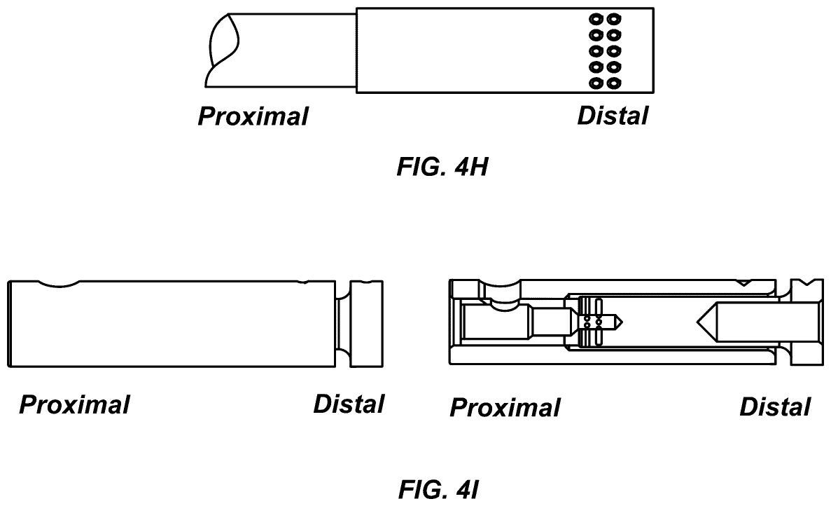

FIGS. 4A-4C illustrate an example of a 360 degree rotational turbine diffuser suitable for use within conjunction with cryo-ablation catheters. As shown in FIG. 4A, representative diffuser assembly 36 is comprised of a non-rotating discharge core 404, a rotatable assembly 400 and a retainer 401. As discussed here generally with reference to implementations in which rotatable assembly 400 is comprised of a rotatable discharge disk 403 mated to a discharge cover 402, rotatable assembly 400 is secured to the non-rotatable discharge core by means of a retainer 401 so as to be freely rotatable about an internal portion 404b of the non-rotating discharge core 404. The rotatable assembly 400 (as well as any other rotatable assembly described herein) can have a full rotational range of 100 RPM to 100,000 RPM, with, for example, a preferred range of 5,000 RPM to 50,000 RPM. Retainer 401 can be secured by gluing the proximal side to 404b and 30 using an adhesive. In one implementation, Epoxy (Loctite M-121) is used; however, many other commercially available adhesives are also suitable. In other implementations, rotatable assembly 400 can also be realized using fewer or greater numbers of subcomponents, which can be matted together as needed by a varying combination of techniques such as adhesives, mechanical locking features, press-fit, and/or the like. While depicted generally as a ring shaped retainer 401, located at the proximal end of diffuser 36, and of like diameter to rotatable assembly 400, retainer 401 can be implemented by a variety of other mechanisms, including a washer and retaining pin arrangement, ball and detent implemented between rotatable assembly 400 and discharge core 404, retaining rings, e.g., E-clip rings, poodle clip rings, crescent rings, snap rings, grip rings, internally mounted axial rings, and others. In some implementations, the retainer 401 can be implemented using crimping, as well as any of a variety of mechanical locking features, which can provide an interference fit to 404, i.e., press-fit, or threaded to a stop as will be evident to the skilled artisan.

Now with reference to FIG. 4B, discharge core 404 is non-rotational during operation and comprised of an external portion 404a at the distal end and an internal portion 404b running throughout the diffuser 36 to the proximal end. Disk 403 includes a plurality of orifices 40 formed by cavities 49 within the disk 403 and surfaces of cover 402 and core 404 in the diffuser assembly. The orifices 40 shown in FIGS. 4A-4C may have a diameter of about 0.508 mm to 1.524 mm (0.020 inches to 0.060 inches), typically about 1.016 mm (0.040 inches). However, in some implementations, the discharge sizes should typically range from 0.0254 mm to 1.524 mm (0.001 inches to 0.060 inches) and typically between 0.127 mm to 0.381 mm (0.005 inches to 0.015 inches). In one embodiment, the devices have a discharge diameter of 0.2032 mm (0.008 inches). In operation, refrigerant is conducted via internal pathways 41 in core 404 to cavities 49 to be dispensed by orifices 40 radially outward in a stream, causing the discharge disk 403 to rotate about the internal portion 404b of the discharge core 404, thereby broadcasting the refrigerant onto an inner surface of the balloon 24 in a 360 degree discharge pattern. The diffuser configurations of FIGS. 4A-4C can replace existing fixed diffusers of an existing cryo-ablation catheter as well as form the basis for a new cryo-ablation catheter system. Implementations of this 360 degree rotational diffuser, in conjunction with the existing cryo-ablation catheters, can create a full 360 degree cylindrical ablations.

The discharge core 404 performs several functions in the diffuser assembly 36. For example, the discharge core 404 provides a structural element for the assembly allowing the connection to the distal and proximal diffuser guides (not shown in FIG. 4B for clarity's sake). These guides act to center the diffuser within the balloon 24. This centering helps to maintain uniform distance circumferentially between the target tissue and the diffuser. It also is part of the overall structure of the balloon assembly. It provides a place proximally for the delivery line, which is the conduit for the liquid cryogen, in this particular use nitrous oxide. The delivery tube 30 is secured into place inside the lumen 43 positioned at the proximal end of discharge core 404 e.g., by means of adhesive, such as an epoxy-type adhesive, or other bonding means.

With continuing reference to FIG. 4B, liquid cryogen (refrigerant) which discharges from the distal end of delivery tube 30 flows through a proximal channel in the discharge core 404 until it is redirected radially outward by internal pathways 41 into cavities 49 in the discharge disk 403/discharge cover 402 assembly. The distal side of the discharge core 404 has a void space 46 for receiving member 51 which acts as a central rail which interfaces with the distal end of the balloon 24 assembly with the delivery channel guide.

The discharge disk 403 and discharge cover 402 form a rotating assembly 400 by aligning the outside diameter with the discharge cover 402 and fixing it to the discharge disk 403 by means of adhesive, such as an epoxy-type adhesive, or other bonding means. In various implementations, Loctite 4014, Loctite 4061 and Loctite M-121 HP are used; however, many other commercially available adhesives are also suitable. Once fixed together, the inner cavities 49 (chambers) of the discharge disk 403 are fully defined viaways. The inside diameter (ID) of the discharge disk 403/discharge cover 402 rotating assembly 400 fits over the shaft formed by the internal portion 404b of the discharge core 404 as shown in FIG. 4B. In alternative implementations, rotating assembly 400 can be constructed as a single workpiece, such as by 3D printing, molding, casting, or the like.

Now with reference to FIG. 4C and with continuing reference to FIG. 4B, distal to the lumen 43 for receiving the delivery tube 30 in the discharge core 404 are internal pathways 41 that form the axial and radial flow path for conducting the liquid cryogen. The liquid cryogen then flows through the radial holes of the internal portion 404b of the discharge core 404 and into the cavities 49 (chambers) of the discharge disk 403/discharge cover 402 rotating assembly 400 from which it is vented via discharge channels 37 to orifices 40.

The liquid cryogen flowing into and out of the chambers 49 causes the discharge disk 403/discharge cover 402 rotating assembly to rotate about the “shaft” formed by internal portion 404b of discharge core 404. While the discharge disk 403/discharge cover 402 rotating assembly 400 is rotating, the liquid cryogen discharges from the orifices 40 of the discharge disk 403/discharge cover 402 rotating assembly 400. The liquid cryogen then travels to the target surface (i.e., the interior surface of balloon 24). As the liquid cryogen comes in contact with the target surface the liquid cryogen changes phase from a liquid to a gas removing energy from the targeted surface and area. This loss in energy causes the temperature of the targeted area to drop to a lethal level.

As the discharge disk 403/discharge cover 402 rotating assembly 400 rotates and discharges the liquid cryogen, the full diffuser assembly 36 translates axially at a selectable speed. The slower the translation speed, the higher the dosing, i.e., the slower the translation speed, the greater is the amount of cryogen that is applied. Conversely, the faster the translation speed, the lower the dosing. This translation during the discharge allows the liquid cryogen to impinge on a full circumferential surface of a defined length such as a section of an esophagus, a duodenum, bowel, etc. In this way a full section of the organ can be more easily and quickly treated.

### Operation

When assembled into a cryo-catheter, the diffuser can be operated with the use of a standard cryo-catheter controller, foot pedal, and liquid cryogen, typically nitrous oxide, cartridge for example. The cryo-catheter is connected into the distal end of the controller. The controller supplies and regulates the flow of liquid cryogen. The controller accepts inputs from the user, provides user feedback, monitors/controls various aspects of pressure, cryogen flow, and diffuser translation speed. The controller is connected to the foot pedal via a power/communication cord. The foot pedal supplies electrical power to the controller as well as allowing the user to control the movement of the diffuser distally and proximally, initiate the release of the cryogen, and stop the release of cryogen. The foot pedal is plugged into a standard outlet which provides power to the foot pedal and controller. Installed into the controller is a source of cryogen. This source is in the form of a pressurized cartridge with liquid cryogen, typically nitrous oxide.

The dosing is set by the user with the controller's touch screen interface. The dosing is quantified by a rate of axial travel. For some tissue ablation applications this speed is typically in the range of 0.5-1.5 mm/sec (0.0197 to 0.059 inches/sec). However different types of tissue with different thermal properties may require higher or lower translation speeds. The length of travel is also set by the user and is typically 1-3 cm (0.394 inches to 1.181 inches). Again, however, this length can vary depending on the specific application. The flow rate of cryogen is established by the pressure of the source, such as a temperature controlled pressurized cryogen cartridge and the diameter/length of the cryogen delivery line.

To deliver a treatment to a specific tissue, the cryo-catheter is placed with the diffuser at the tissue to be targeted. This is typically accomplished via the working channel of a flexible scope. In some cases, it may be desirable to not use the working channel of a scope and place the catheter by other methods. Various means to maintain the position of the diffuser can be employed, such as a balloon incorporated onto the distal end of the catheter surrounding the diffuser. Once the balloon has been placed and inflated at the target tissue, the axial location of the diffuser can be adjusted and aligned as needed. This is achieved under direct visualization with the scope and depressing the forward/backward switches of the foot pedal.

With the translation speed set via the controller's touch screen, the treatment cycle can be initiated by depressing and holding the treatment switch on the foot-pedal. Liquid cryogen will be released from the cartridge, through the controller, into the catheter's delivery line and to the diffuser. The configuration of the diffuser causes rotational forces to be developed with the passing of the cryogen through the diffuser. These forces create the rotation of the discharge orifice(s). With the discharge orifice(s) spraying cryogen in a 360-degree ring at the target tissue, the diffuser assembly is translated axially. This combination creates a 360-degree cylindrical ablation at the desired dosage for the set length.

Once completed, the balloon containing the diffuser can be deflated and diffuser relocated as desired.

FIG. 4D shows an alternative embodiment in which orifices are disposed on both sides of the discharge disk of a rotational diffuser. As shown by FIG. 4D, orifices 40 can be provided on both sides of the discharge disk 403. A second discharge cover 405 is fitted over internal portion 404b to close the viaways of the second (distal) side of the discharge disk 403. An advantage to providing orifices on both sides of the discharge disk is that when each orifice discharges cryogen for the full 360-degrees of the circumference, a more uniform distribution of cryogen can be provided. Of course, providing orifices on both sides of discharge disk 403 requires additional machining to fabricate and an additional part, second discharge cover 405, to form the finished diffuser assembly.

FIG. 4E shows another embodiment in which tubes are inserted in viaways to reduce orifice size a rotational diffuser. As shown by view 4E-1, a small ID tube 460 can be placed and sealed inside one or more of the discharge channels 37 venting the chambers 49 of discharge disk 403 with a goal to reduce the functional size of the orifice(s) 40. The primary advantage is that the size of the orifices 40 can be made much smaller to allow for a more targeted stream of liquid cryogen. Decreasing the orifice size increases the core pressure helping to ensuring a full capacity of cryogen is delivered by each orifice 40. One implementation leverages a precision readily available off the shelf part allowing wider machining tolerances. There are a number of off the shelf tube sizes available which would not be possible to achieve by machining. Of course, implementations of this approach may require an additional part to be placed into the discharge disk 403.

FIG. 4F shows a further embodiment in which the diffuser includes a helical cut element 407 that is free to rotate with passage of the cryogenic fluid exiting via slot 409. The primary advantage is a relatively simplistic design. The disadvantage is the high sensitivity to fits and tolerances of relatively small parts in order to achieve rotation of the inner member.

FIG. 4G shows an alternative embodiment using a non-rotational 360-degree orifice with a proximal helical cryogen flow path. This implementation can be achieved without rotational components. Note: the depression on the proximal end is for manufacturing purposes to enable application of cryogenic ablative and/or adhesive. The primary advantage is that it requires only 2 parts and eliminates the alignment sensitivity between the two parts.

FIG. 4H shows a further alternative embodiment using non-rotational multiple discharge orifices oriented in a 360-degree arrangement at the distal end of cryogen delivery tube. The primary advantage is the simplicity of the design and fabrication.

FIG. 4I shows a still further alternative embodiment using a non-rotational diffuser having a full circumferential 360-degree orifice tip. This implementation can be achieved without rotational components. Note: the depression on the proximal end is for manufacturing purposes to enable application of cryogenic ablative and/or adhesive. The primary advantage is a full 360-degree spray is achieved.

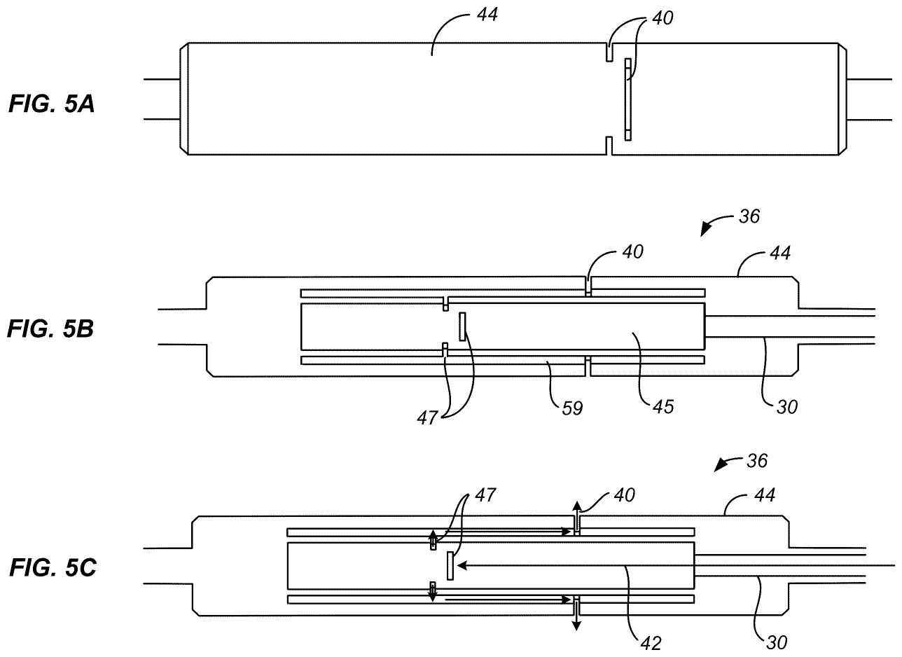

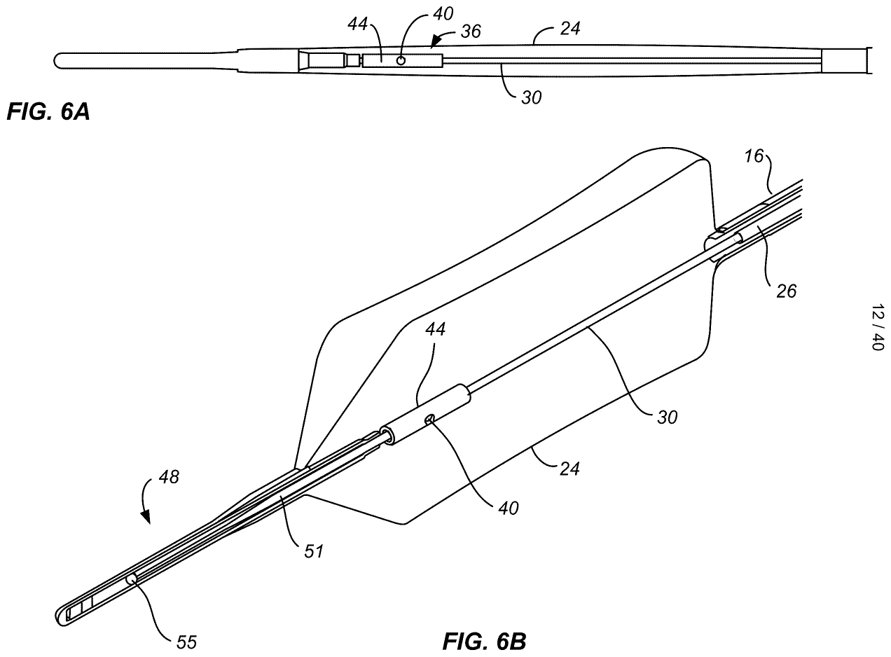

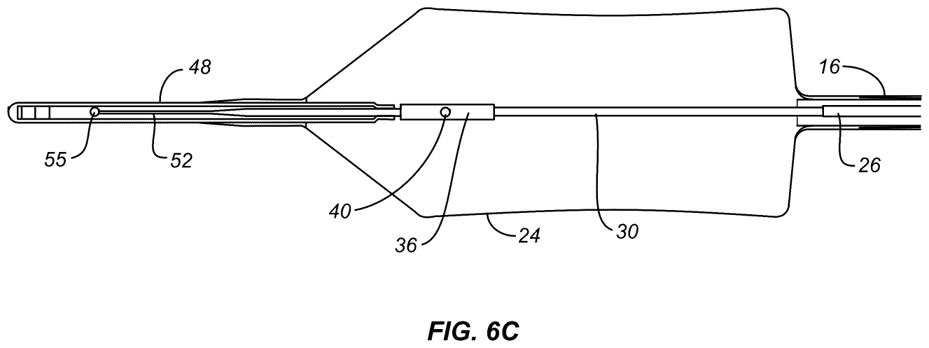

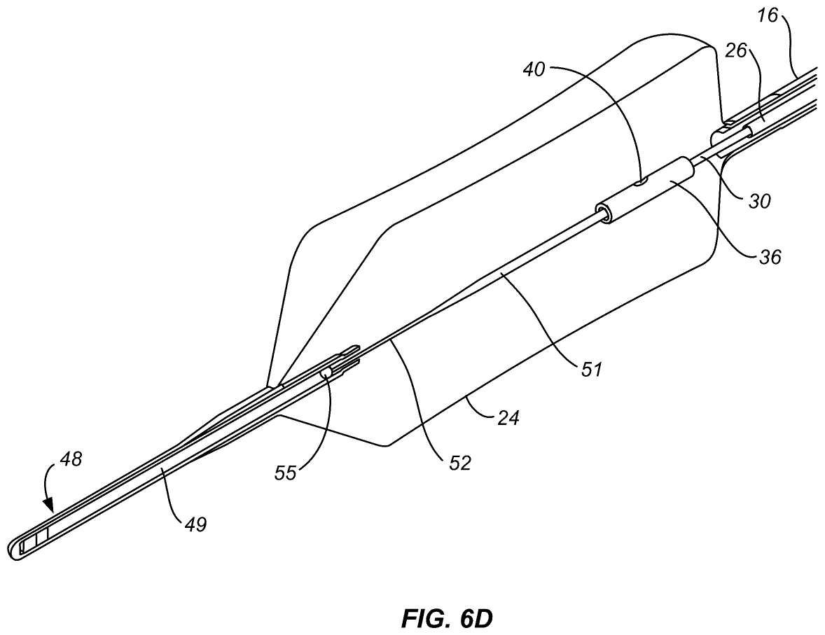

As shown in FIGS. 5A-5C , an alternative example of diffuser 36 that is non-rotational is shown. The outer diffuser tube 44 of the diffuser 36 of FIGS. 5A-5C has a number of nozzle ports 40 arranged to direct refrigerant in a generally 360° rotary pattern in contrast with the limited rotary pattern created by the single nozzle port 40 shown in FIGS. 2A-2C and 6A-6D. The internal flow paths, discussed below with reference to FIGS. 5B and 5C, can be the same with both embodiments. The diffuser 36 comprises a hollow internal cavity 45 fluidly connected to the delivery tube 30. Cavity 45 has, in this example, four lateral passageways 47 extending from internal cavity 45 to a cylindrical cavity 59. Nozzle ports 40 extends through the outer surface of outer diffuser tube 44 and into cylindrical cavity 59 to permit refrigerant to flow as indicated by the refrigerant path 42 in FIG. 5C. This allows refrigerant supplied from a refrigerant fluid source in the handle assembly to be sprayed on the interior wall of the balloon 24. The nozzle ports 40 may be comprised of one or more slits located around the outer diffuser tube 44. The slits may be stacked in multiple rows to allow openings in the wall at all radial position, for example in embodiments where spray is to be delivered 360 degrees. In embodiments the desired delivery angle of the spray may be less than 360, for example 45 degrees, 90 degrees or 180 degrees. In these embodiments the nozzle ports 40 will be sized and positioned to deliver the desired angle of spray. In embodiments the nozzle ports 40 includes slits with a preferred height of 4 thousandths of an inch, but may range from 0.0254 mm to 0.254 mm (0.001 inches to 0.010 inches). The path 42, as shown in FIG. 5C, is configured to allow for even distribution of refrigerant by the point the refrigerant is at the nozzle port end of the diffuser 36 so that the pressure is equal radially around the inner cavity. The single, circular nozzle port 40 shown in FIGS. 6A-6D may have a diameter of about 0.508 mm to 1.524 mm (0.020 inches to 0.060 inches), typically about 1.016 mm (0.040 inches). Additionally, the single port shape is not limited to a circle, but may be other shapes including ellipses and rectangles. The diffuser 36 illustrated in any of the figures described herein can be the 360 degree rotational turbine diffuser described above with reference to FIGS. 4A-4I, even if diffuser 36 is illustrated differently, such as in FIG. 6A.

The balloon 24 is expandable and collapsible and is mounted to the distal end of the catheter shaft 16. FIG. 2A, shows a schematic of the balloon 24 in a deflated tension state, and FIGS. 2B and 2C, show the balloon 24 in an inflated state. Balloon 24 can be an elastic material, such as polyurethane, and can have an operating diameter in the range of 4 mm to 60 mm (0.157 inches to 2.362 inches), with, for example a preferred range of 6 mm to 40 mm (0.236 inches to 1.5748 inches) when inflated with less than 3.45 kPa (5 psi). Balloon 24 has an inner surface defining a balloon interior. In embodiments, the balloon 24 includes a tapered distal end secured to a flexible tip 48. During operation, refrigerant flows out through one or more nozzle ports 40 of the diffuser 36 generally radially outwardly to create refrigerant spray directed at a target site along the inner surface of the balloon 24. The target site of the inflated balloon is in contact with tissue and the delivery of refrigerant typically causes cryogenic ablation of tissue abutting target site of the balloon 24. In embodiments, the target site is larger than the area of spray delivery to the interior wall of the balloon 24 and the diffuser 36 translates along the length of the balloon and/or rotates within the balloon 24 while spraying to deliver refrigerant to the entire target site. Noteworthy is that the rotational turbine diffusers described herein above with reference to, at least FIGS. 4A-4I, have structures such that it is not necessary for the entire diffuser 36 to rotate, in contrast with other configurations of diffuser 36 as described with reference to FIGS. 6A-6D and so forth. For example, it is not necessary for the deliver tube 30 to rotate in rotational turbine diffusers described in FIGS. 4A-4I. The portion of the balloon capable of receiving refrigerant spray and shaped to be capable of contacting tissue is referred to as the working length of the balloon. In embodiments, the working length of the balloon 24 includes straight wall portions, as shown in FIGS. 2B and 2C. The balloon may further include tapered wall portions that usually do not contact tissue or receive refrigerant spray, as shown in FIGS. 2B and 2C. In embodiments, as shown in FIGS. 6B-6D, balloon 24 can have an hourglass shape with its smallest diameter, its waist, being smallest at a position spaced apart from either end. The balloon with this configuration can be selected because it facilitates properly locating and cryogenically ablating target tissue which extends inwardly within the hollow body structure being treated. Examples of such generally extending tissue include tissue at the sphincter between the esophagus and stomach, and other tissue structures and shapes which are difficult to conform to using a balloon having, for example, a cylindrical outer surface. Therefore, during use a balloon can be selected having a non-cylindrical shape, such as an hourglass shape, which facilitates conforming the outer surface of the balloon to the tissue being treated. Balloons having shapes other than the moderate hourglass shape shown in FIGS. 6B-6D could be selected depending on the tissue being treated. For example, a balloon having two reduced diameter, waist portions could be chosen; the waist portions could have the same or different diameters. In embodiments the balloon 24 may include strain gauges used as input into a controller 50, which is discussed below.

The balloon 24 is shown in detail in FIGS. 6A, 6B, 6C and 6D. The flexible tip 48 is configured to assist in guiding the balloon end of the catheter while inserting the distal end of catheter 12 into a device, such as an endoscope, or into a bodily passage, such as an esophagus. For example, endoscopes commonly have a kink in the port at which the catheter is inserted. The flexible tip 48 is more flexible than the delivery tube 30 and prevents damage to the delivery tube 30 and balloon 24 during insertion of the catheter 12. For example, during initial insertion the flexible tip 48 may encounter an obstacle causing it to bend a substantial amount. This amount of bending may cause damage to the delivery tube 30 and render it inoperable. Therefore, the flexible tip 48 may act as a sacrificial bending point which may be caused to bend a large amount during initial insertion and not have an effect on the operability of the overall device because the delivery tube 30 will be able to pass by the obstacle with a more gentle bend because the flexible tip 48 is further along the path of insertion and able to guide the remainder of the catheter 12. Further, the flexible tip 48 may prevent damage to tissue in the body if during insertion the tip impacts tissue.

Flexible tip 48 includes a cylindrical cavity 59 slidably housing a delivery tube extension 51. Extension 51 is affixed to and extends from diffuser 36. Extension 51 is preferably made from a flexible material which resists kinking, such as nitinol. As shown best in FIG. 6D, extension 51 has a reduced diameter distal portion 52 to enhance the flexibility of the flexible, atraumatic tip 48. One way to create the reduced diameter portion 52 is through the use of centerless grinding. As shown in FIG. 6B, the flexible tip 48 includes a rounded end 55.

Translating the delivery tube 30 toward the flexible tip 48 can cause rounded and 55 to contact the distal end of flexible tip 48. This can cause the balloon 24 to stretch in tension to the collapsed, minimum diameter position shown in FIG. 2A. The uses and benefits of this stretched position will be discussed in detail below.

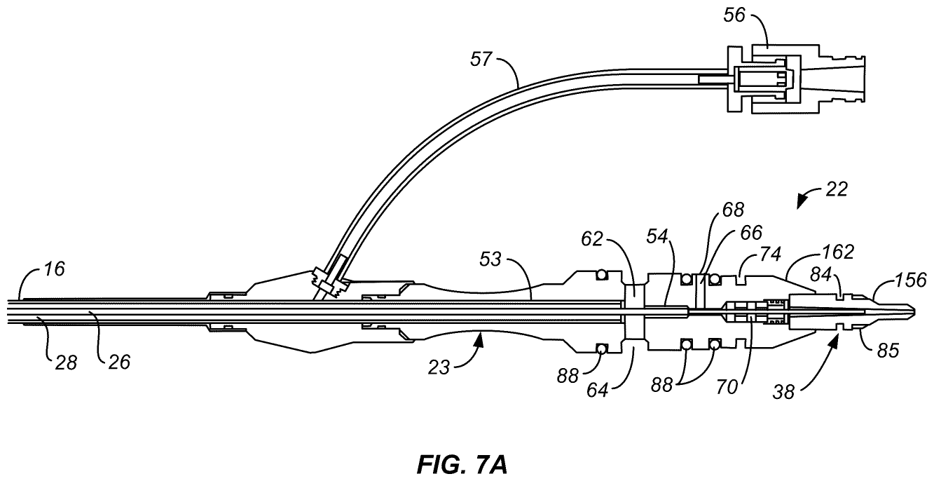

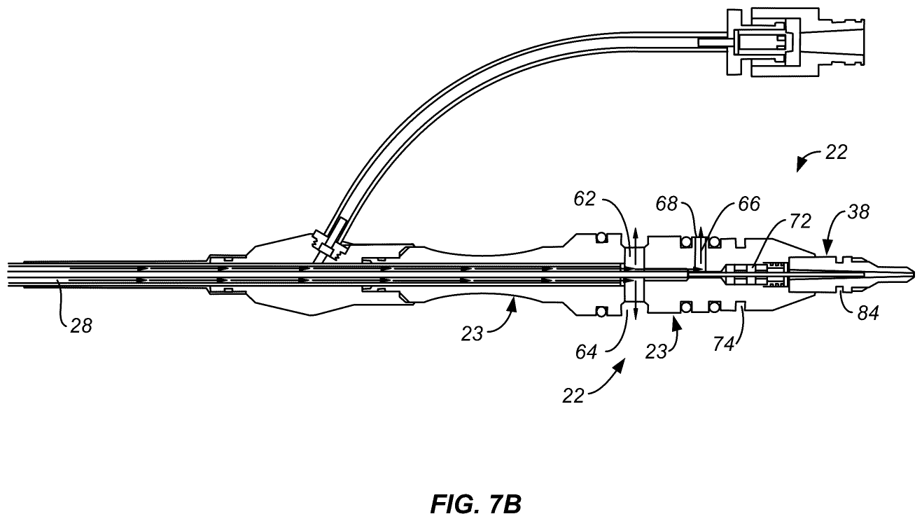

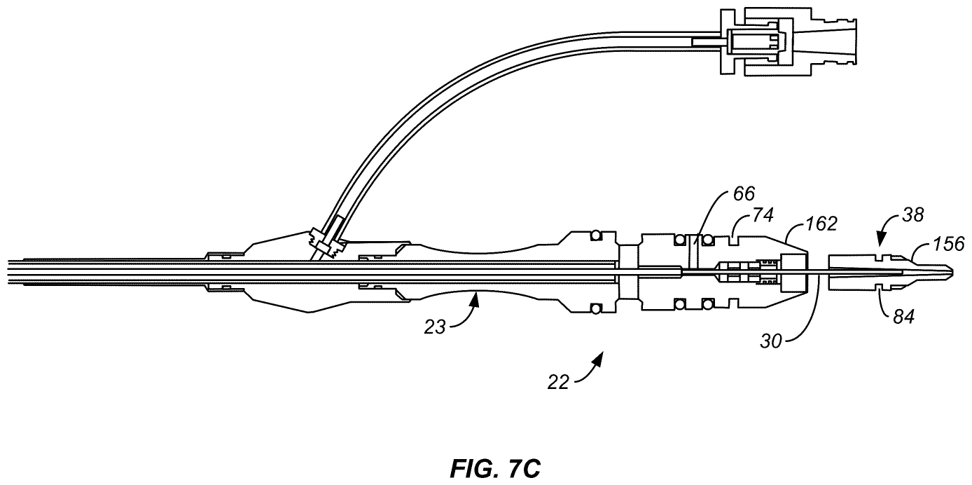

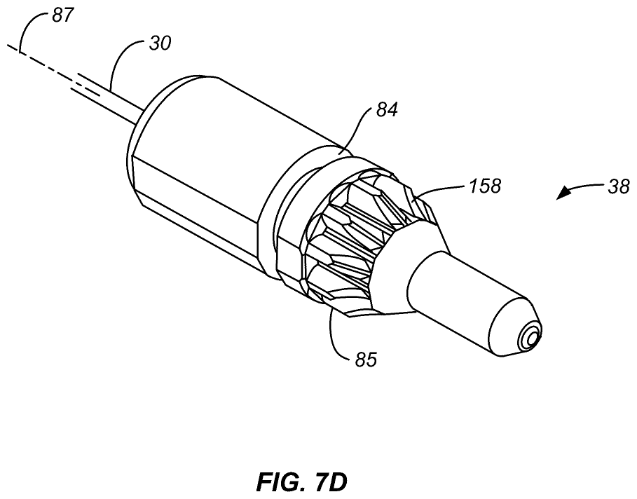

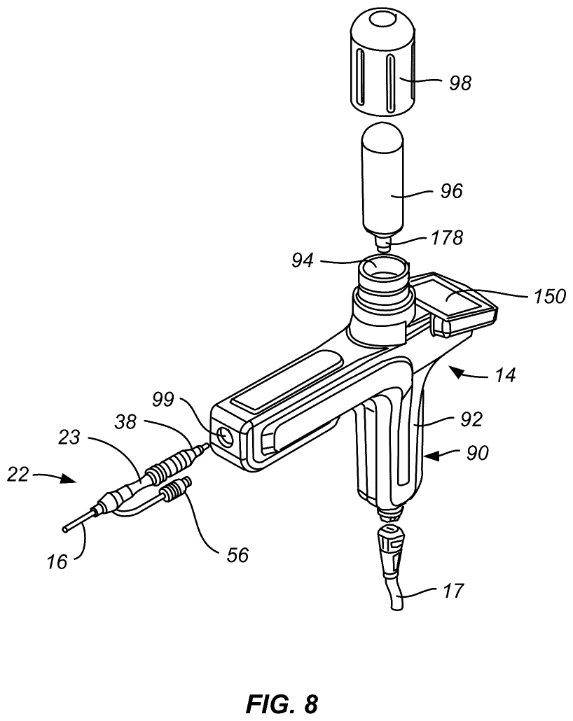

FIGS. 7A-7C show the connector 22. The proximal end of the catheter shaft 16 and the pressure detecting tube 26 are affixed to positions 53 and 54 within the connector body 23. The connector 22 includes an exhaust passage 62 that fluidly couples the exhaust lumen 28 to a radial exhaust port 64 on the exterior of the connector body 23. The connector body 23 includes a pressure detecting passage 66 that fluidly couples the pressure detecting lumen 32, see FIGS. 3B and 3C, to a radial pressure detecting port 68 on the exterior of the connector body 23. The connector body 23 further includes a central passage 70 that the delivery tube 30 passes through between the pressure detecting passage 66 at the proximal end of the connecter 22. The delivery tube 30 is free to translate and rotate in the central passage 70 as well as pressure detecting tube 26. The central passage 70 is separated from the pressure detecting passage 66 by one or more seals 72 that allow the delivery tube 30 to translate and rotate in the pressure detecting tube 26 and pressure detecting passage 66 but prevents gas from the pressure detecting passage 66 being leaked out the central passage 70. Connector body 23 also includes a circumferentially extending body locking slot 74 used to secure connector body 23 to handle assembly 14. This will be described in more detail below.

Balloon 24 can be deflated by connecting the interior of the balloon 24 to the ambient atmosphere through exhaust lumen 28 for the passage of gas into handle assembly 14 and then out to the ambient atmosphere. Doing this does not necessarily fully collapse the balloon. A syringe, or other appropriate device, can be fluidly coupled to the exhaust lumen 28 within connector body 23 through a syringe coupler 56 connected to connector body 23 by tubing 57. Syringe coupler 56 includes a one-way valve which opens only when a syringe, or other vacuum/pressure application structure, is mounted to syringe coupler 56. In addition to removing gas from the balloon, a syringe can be used to expand the balloon, or expand and contract the balloon, such as during placement of the balloon.

FIG. 7D is an enlarged perspective view of the plug 38 with delivery tube 30 passing through the plug and extending distally therefrom. As discussed above, delivery tube 30 is affixed to plug 38, such as with an epoxy type adhesive or other adhesive, or other bonding means. Plug 38 has a circumferentially extending plug locking slot 84 used to pull plug 38 proximally and push plug 38 distally along the delivery tube axis 87. This causes corresponding movement of the delivery tube 30 and diffuser 36. Plug 38 also has gear teeth 85 used to allow plug 38, and delivery tube 30 therewith, to be rotated about the delivery tube axis 87.

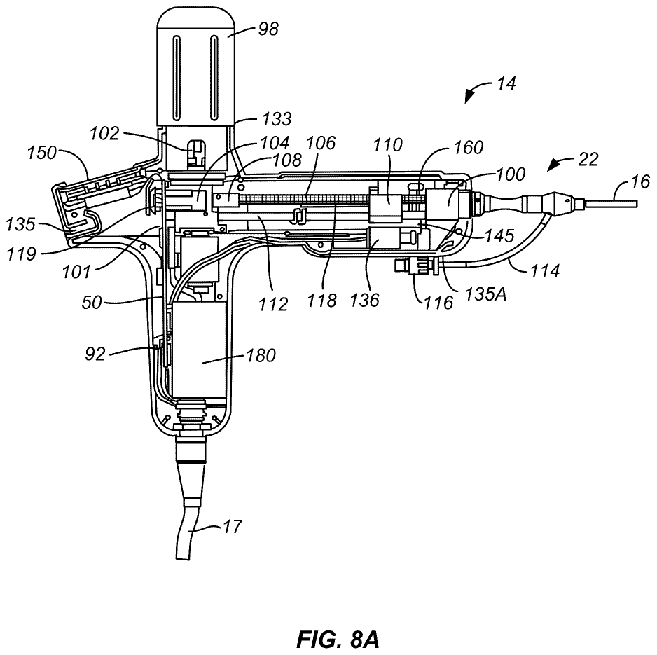

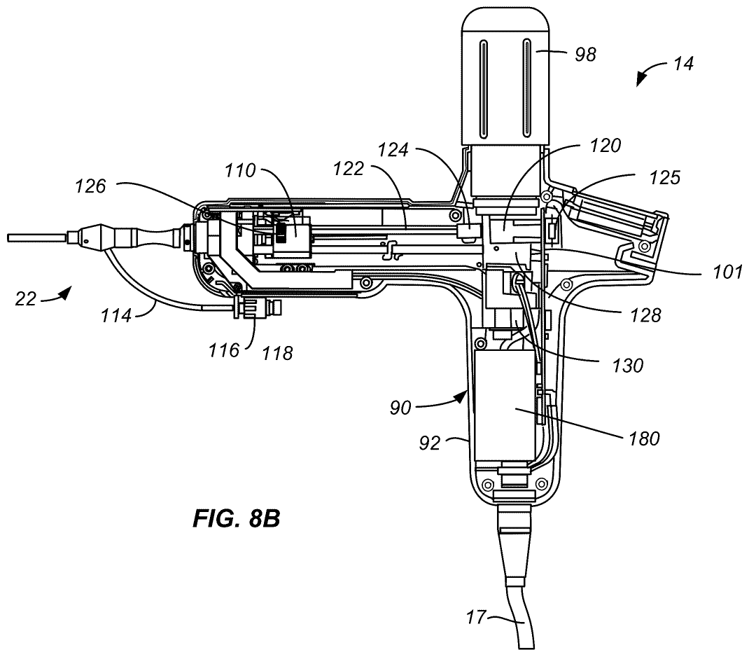

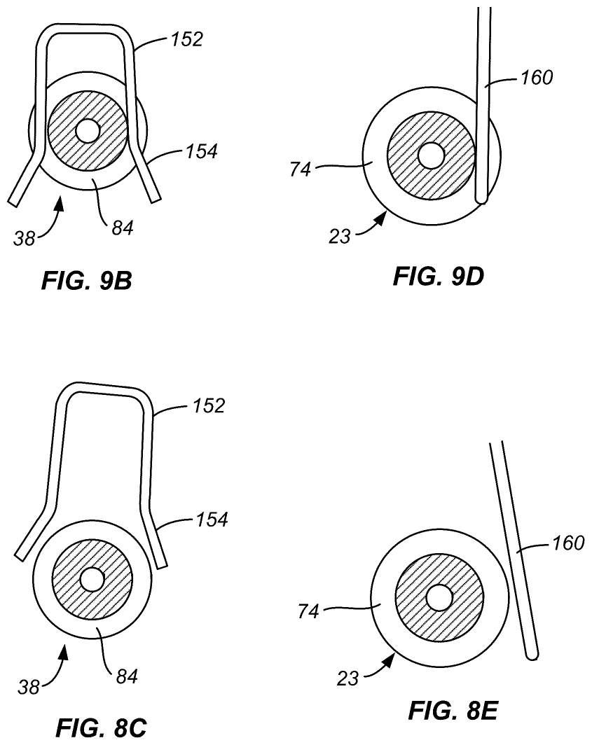

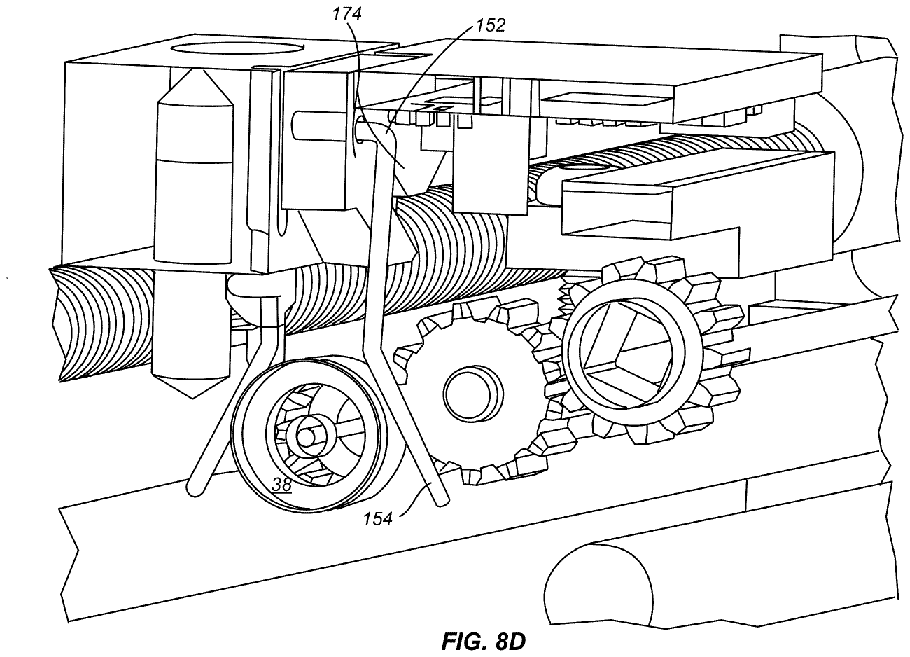

FIG. 8 is a partially exploded isometric view of portions of ablation assembly 10. Handle assembly 14 is shown to include a handle assembly body 90 having a handgrip portion 92 to which power and control line 17 is attached. Body 90 also includes a hollow, externally threaded tower 94 used to receive a refrigerant source 96, typically a nitrous cartridge with a preferred size of ˜50 mL containing about 36 grams of nitrous oxide. A hollow, internally threaded cap 98 secures refrigerant source 96 within handle assembly body 90. Handle assembly 14 also includes a connector receptacle 99, also called first connector element 99, of controller connector 100, see FIG. 8A, for receipt of connector 22.

FIG. 8A is a right side view of the structure shown in FIG. 8 in a partially assembled form with the right side of the handle assembly body 90 removed to show internal components. Controller 50 shown mounted to a main printed circuit board 101. Connector 22 is shown in the process of being fully inserted into connector receptacle 99 to be placed in the load position. Assembly 14 includes a heater 102 used to heat nitrous cartridge 96 when needed. A linear drive motor 104 is connected to a rotatable, threaded shaft 106 by a threaded shaft coupler 108. Threaded shaft 106 passes through and is threadably connected to a traveler 110, also referred to as the traversing member, resulting in the linear, axial movement of the traveler. Traveler 110 is supported by and guided by a pair of bearing shafts 112, 113 along which traveler 110 slides. Bearing shaft 113 is shown in FIG. 8B. Extending from connector 22 is a balloon inflation/deflation line 114 terminating at the connector 116, typically configured to attach to a syringe. The syringe is typically used when desired to completely deflate balloon 24. Refrigerant from cartridge 96 passes through a delivery line 118 terminating at traveler 110. Delivery line 118 is a flexible delivery line and can be looped around bearing shafts 112, 113 without kinking to accommodate the linear, axial movement of traveler 110. The rotation of linear drive motor 104 is monitored by controller 50 through the use of a counter wheel 119, having light and dark segments, and an appropriately located light sensor to monitor the rotation of counter wheel 119.

FIG. 8B corresponds to FIG. 8A but illustrates the left side of the structure. Handle assembly 14 includes a rotation motor 120 connected to a rotation shaft 122 by a rotation coupler 124. Rotation of rotation motor 120 is monitored by controller 50 through the use of a counter wheel 125 and an appropriately located light sensor in a manner similar to that used with linear drive motor 104. Rotation shaft 122 has, in this example, a square cross-sectional shape; other rotationally driving shapes can also be used. Rotation shaft 122 passes through traveler 110 a manner which allows shaft 122 to freely rotate within traveler 110 and allows traveler 110 to move freely in a linear, axial manner. A drive gear 126 is bracketed by traveler 110 to move with the traveler. Drive gear 126 is rotationally coupled to rotation shaft 122 so that rotation of shaft 122 causes drive gear 126 to rotate. Drive gear 126 is slidably mounted to rotation shaft 122 so that the drive gear slides along rotation shaft 122 as traveler 110 moves in its linear, axial manner. As will be explained below, when connector 22 is mounted to handle assembly 14, rotation of shaft 122 causes the plug 38 and delivery tube 30 therewith to rotate.

Refrigerant from refrigerant source 96 passes through a manifold 128 (FIG. 8B) under the control of the refrigerant controlling delivery solenoid 130, through a delivery line coupler 129, see FIG. 9, and into delivery line 118. The pressure within refrigerant source 96 is monitored by controller 50 through a pressure transducer 141. The operation of delivery solenoid 130 can be controlled by the operator using foot pedal assembly 15. As will be discussed in more detail below, tapping refrigerant delivery foot pedal 132 typically provides enough refrigerant to expand balloon 24 and provide visualization of the location of the refrigerant port. Application of refrigerant to the inner surface of balloon 24 to ablate tissue can be controlled manually by the user pressing on foot pedal 132. The system can be programmed to provide a set period of refrigerant delivery depending on the particular therapy, through either a regulated time such as 2 to 20 seconds or regulated translation speed such as 0.5 to 1.5 mm/sec (0.0197 to 0.059 inches/sec). and is preferably programmed to limit the maximum length of time for refrigerant delivery to a target treatment site to, for example, 10 seconds. Foot pedal assembly 15 also includes a foot-actuated deflation button 134 which allows the operator to deflate the balloon, typically placing the balloon interior of the balloon at atmospheric pressure. Pressing deflation button 134 actuates an exhaust solenoid valve 136, see FIG. 9A, which is connected to exhaust passage 62 (FIG. 7B) of connector body 23 by a passageway 138. The exhaust from the balloon 24 through the actuation of exhaust solenoid valve 136 enters the interior of handle assembly body 90. Body 90 has two, redundant ports 133, 135 to help ensure that gases vented into the interior of handle assembly body 90 can escape to the ambient environment regardless of how handle assembly body 90 is being held. Furthermore, if the exhaust valve 136 fails to open or is otherwise occluded, pressure relief valve 145 will actuate, mitigating the risk of balloon over pressurization. See FIGS. 9E and 9F. Pressure relief valve 145 also enables the balloon to remain statically inflated at a regulated pressure of, for example, 19.9 kPa (2.9 psig).

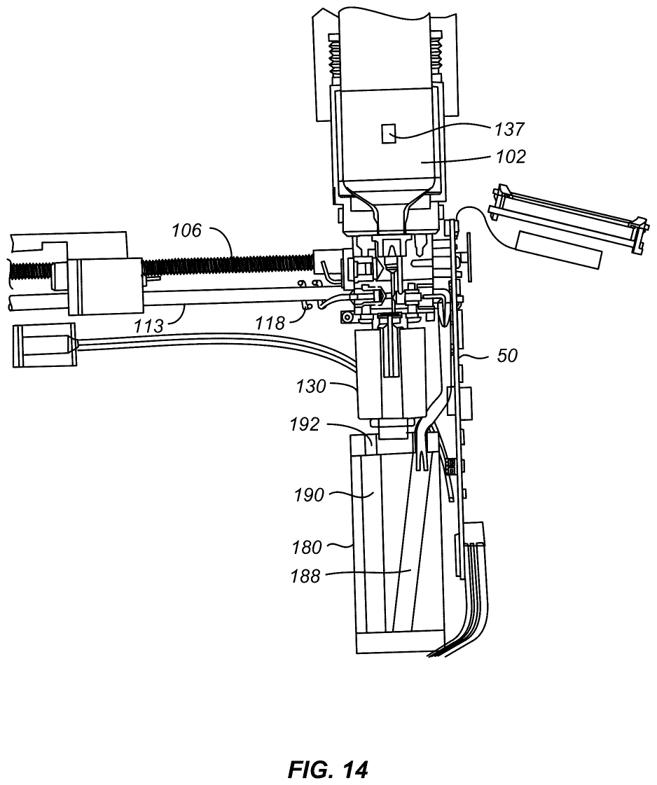

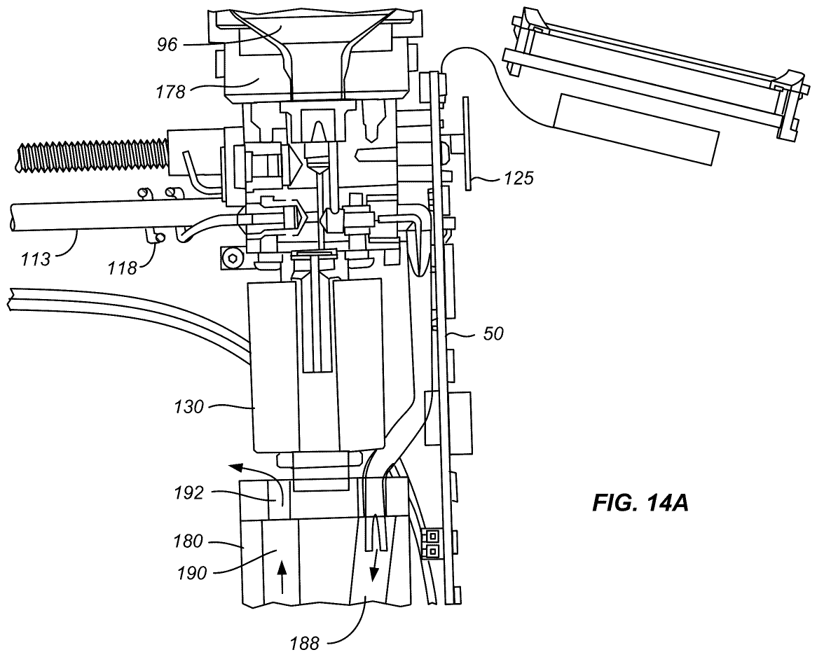

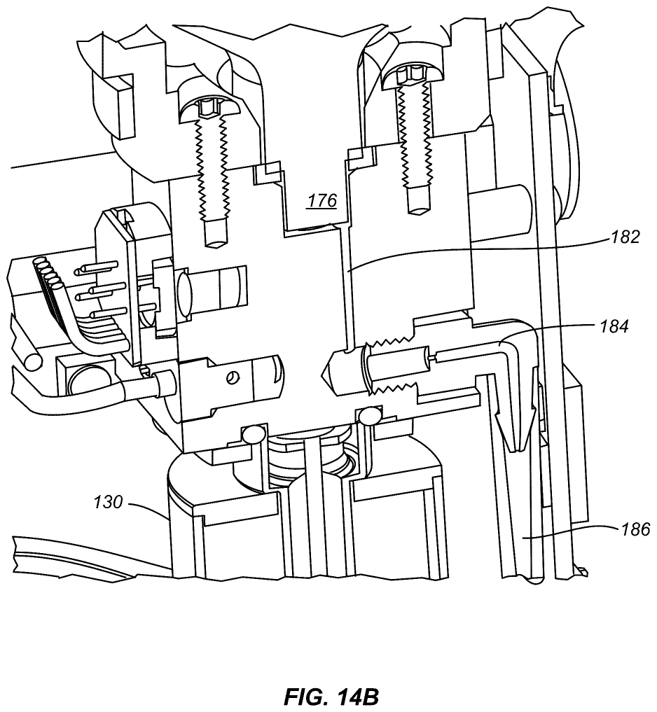

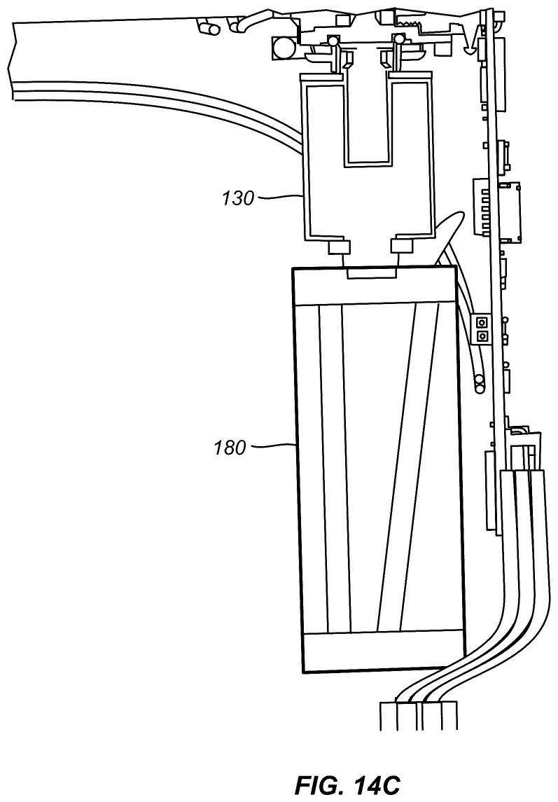

Controller 50 controls and monitors the pressure of the refrigerant within refrigerant source 96 using a temperature sensor such as a thermistor 137, see FIG. 14, and pressure transducer 141, see FIG. 9. Having accurate pressure and temperature information for the refrigerant within refrigerant source 96 allows the system to determine if the nitrous oxide cylinder contains liquid based on the saturated liquid/gas properties of the refrigerant. Additionally, the temperature sensor can be used to detect a malfunction in the heater circuit that may result in overheating and hence over pressurization of the refrigerant cartridge. If overheating is detected, the controller will turn of the heater and/or completely disconnect itself from power.

Foot pedal assembly 15 also includes the left and right movement foot pedals 140, 142. Left and right foot pedals 140, 142 are used to control linear drive motor 104 and rotation motor 120. The user selects which function left and right foot pedals 140, 142 will be used for, that is linear movement or rotational movement. Upon the use of movement mode button 144, foot pedal assembly 15 provides the operator with an indication of which mode has been selected by the illumination of either straight arrows 146, 147, or curved arrows 148, 149. Assuming movement mode button 144 is pressed and straight arrows 146, 147 are illuminated, actuation of left or right foot pedals 140, 142 will cause linear drive motor 104 to operate thus rotating threaded shaft 106 and causing traveler 110 to translate a linear manner. As indicated by the orientation of arrow 146, in this example pressing on left foot pedal 140 causes traveler 110 to move to the left in FIG. 8A thus causing diffuser 36 to move in a proximal direction. Similarly, pressing on right foot pedal 142 causes traveler 110 to move to the right in FIG. 8A causing diffuser 36 to move in a distal direction. This movement can also be preprogrammed and/or limited in the length of travel permitted.

Depressing movement mode button 144 again changes the mode from linear motion to rotational motion. When the system is in the rotational motion mode, counterclockwise curved arrow 148 and clockwise curved arrow 149 are illuminated indicating the direction of rotation of delivery tube 30 and diffuser 36 associate with pressing left and right foot pedals 140, 142. Depressing the left foot pedal 140 provides a signal to rotation motor 120 to rotate rotation shaft 122 in a counterclockwise direction thus causing delivery tube 30 and diffuser 36 therewith to rotate in a counterclockwise direction. Depressing right foot pedal 142 provides a signal to rotation motor 120 to rotate rotation shaft 122 in a clockwise direction thus causing delivery tube 30 and diffuser 36 to rotate in a clockwise direction.

Typical operational parameters for ablation assembly 10 include the following. Translation at a rate between 0.25 mm/sec to 2.5 mm/sec (0.00984 to 0.0984 inches/sec), wherein the rate of translation for therapeutic use is between 0.5 mm/sec and 1.5 mm/sec (0.0197 to 0.059 inches/sec). Rotation can be at a rate between 1 and 10 RPM.

In addition to having arrows 146-149 illuminate, controller 50 could create a visual indication of the selection on an LCD display 150 on handle assembly 14. In addition, an audible indication of the selection can be provided by broadcasting a verbal alert, such as linear movement selected, or by a nonverbal alert, for example a single beep for counterclockwise rotation, and a double beep for clockwise rotation, a long tone for proximal linear movement, and a double long tone for distal linear movement.

FIG. 9B is a simplified illustration showing the relationship of catheter plug locking wire 152 relative to plug locking slot 84 of plug 38 when assembly 10 is in the connector-attachment state of FIGS. 9 and 9A. This state is also shown in FIG. 9C. As plug 38 is inserted through first connector element 99 and into handle assembly body 90, the legs 154 are expanded outward while riding along tapered surface 156 of plug 38, see FIG. 7D, and the tapered leading edges 158 of gear teeth 85. When connector 22 is fully inserted into handle assembly body 90, the legs 154 of catheter plug locking wire 152 will snap into plug locking slot 84 as shown in FIGS. 9B and 9C.

FIG. 9D is a simplified illustration showing the relationship of connector body locking wire 160 relative to body locking slot 74 of connector body 23 when ablation assembly 10 is in the connector attachment state of FIGS. 9 and 9A. As connector body 23 is inserted through connector receptacle 99 and into handle assembly body 90, connector body locking wire 160 is deflected away, FIG. 8E, as it rides along tapered surface 162 of connector body 23, see FIG. 7A. When connector 22 is fully inserted into handle assembly body 90, connector body locking wire 160 will snap into body locking slot 74 as shown in FIG. 9D. Accordingly, examples of ablation assembly 10 provide for the automatic attachment of connector 22 to handle assembly 14.

FIG. 10 illustrates the relationship among drive gear 126, driven by rotation shaft 122, gear teeth 85 of plug 38, and an idler gear 166 coupling the two. Drive gear 126 and idler gear 166 are both mounted to traveler 110 at fixed locations relative to traveler 110, but are free to rotate, and remain engaged as traveler 110 is moved in a linear manner by the rotation of threaded shaft 106. The ends 168 of the gear teeth of idler gear 166 have a V-shaped taper to promote the proper engagement with gear teeth 85 as plug 38 is inserted through first connector element 99 when ablation assembly 10 is in the connector attachment state of FIG. 9.

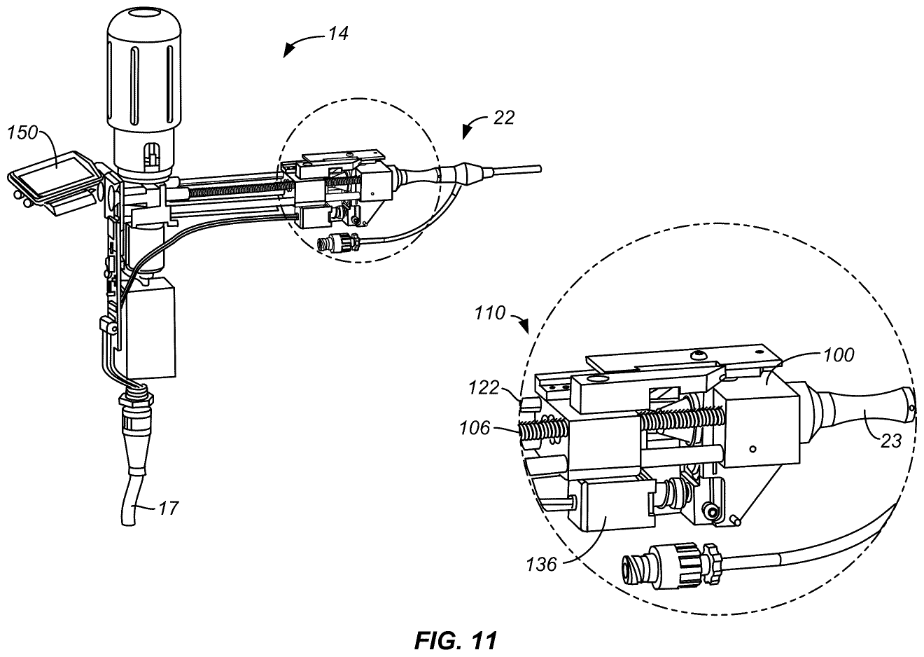

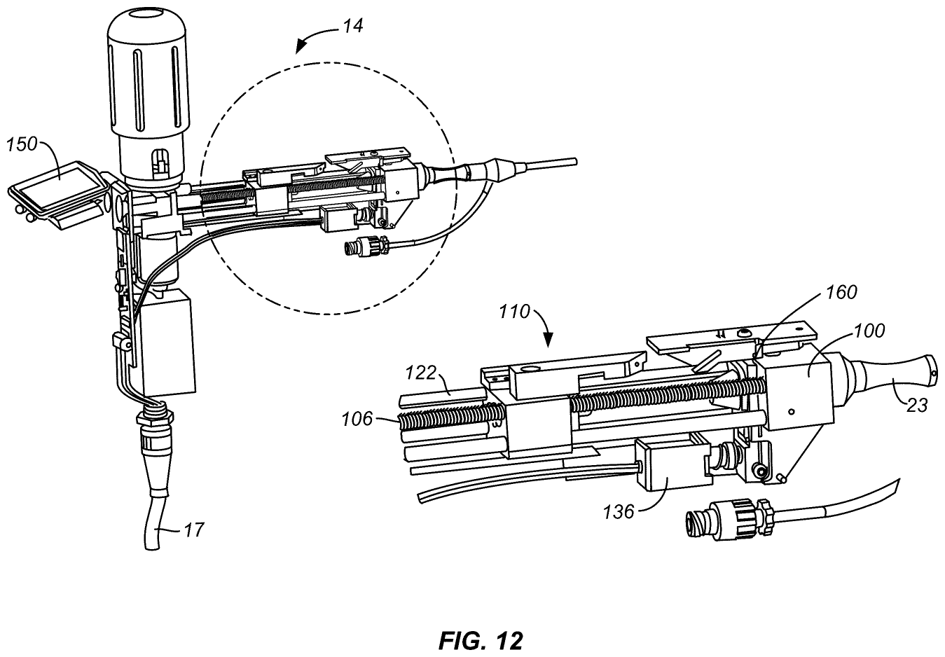

FIG. 11 illustrates a portion of the structure of FIG. 9 after movement of traveler 110 a short distance proximally, to the left in FIG. 11, placing assembly 10 in a distal, typically starting, treatment position corresponding to FIG. 2B. FIG. 12 shows the structure of FIG. 11 after further movement of traveler 110 in a proximal direction, that is to the left in FIG. 12, placing assembly 10 in a proximal, typically ending, treatment position corresponding FIG. 2C. Because refrigerant is sprayed into the interior of the balloon 24 during treatment, the exhaust valve 136 can remain open during the treatment.

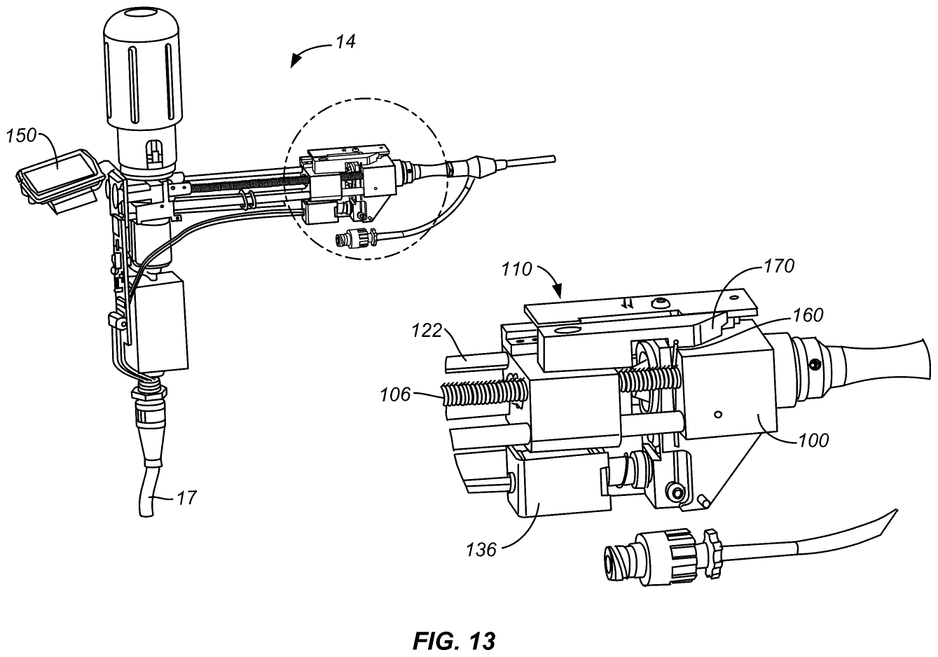

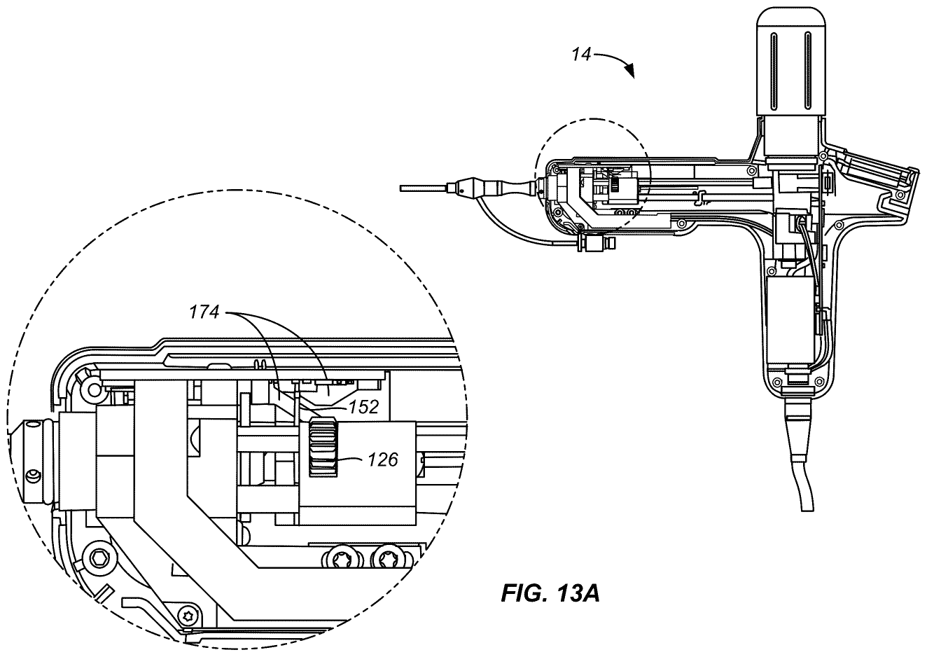

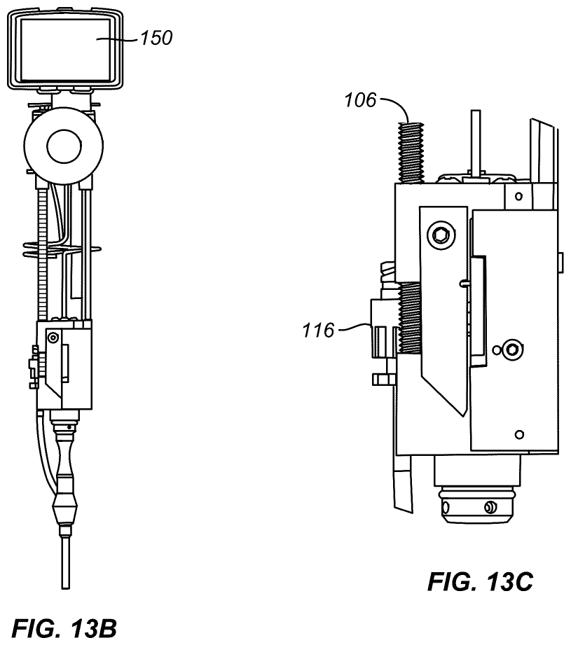

FIGS. 13, 13A, 13 B and 13C show handle assembly 14 in a connector disconnect state with traveler 110 moved in the distal direction, that is to the right in FIG. 13, to a catheter disconnect position. Doing so causes connector locking wire 160, mounted to controller connector 100, be deflected outwardly by the engagement of ramp 170, ramp 170 being a part of traveler 110, so that it moves out of body locking slot 74 as shown in FIG. 8E. Continued movement of traveler 110 to the catheter disconnect position also causes catheter plug locking wire 152, which moves with traveler 110, to be engaged by a ramp 174 causing plug locking wire 152 to move out of plug locking slot 84 from the plug engaged position of FIGS. 9B and 9C to the plug disengaged position of FIGS. 8C and 8D. Ramp 174 is part of controller connector 100. The distal motion of traveler 110 to the catheter disconnection position results in the partial rejection of connector 22 from first connector element 99 of controller connector 100. Connector body 23 is released first, followed by the release of plug 38. Accordingly, examples of ablation assembly 10 provide for the automatic detachment/ejection of connector 22 from handle assembly 14.

When a nitrous cartridge 96 is removed from handle assembly 14, it is important to safely deal with any remaining refrigerant within the cartridge. Handle assembly 14 has a liquid path, shown in FIGS. 14, 14A, 14B, and 14C, for the passage of liquid refrigerant from a region 176 adjacent to the tip 178 to a refrigerant venting chamber 180. Liquid path includes first, second and third portion 182, 184, 186. First portion 182 extends to region 176, and second portion 184 connects first portion 182 to third portion 186. Third portion 186 extends to refrigerant venting chamber 180. The excess refrigerant released from refrigerant source 96, typically nitrous oxide, when the cartridge is removed from handle assembly 14 is typically in liquid form. Refrigerant venting chamber 180 is filled with a foam material, typically open cell polyurethane foam, and has an entrance path 188 and an exit path 190 formed in the foam material. Exit path 190 opens into the interior of handle assembly 14 at an exit port 192. Under the reduced pressure within venting chamber 180, the liquid refrigerant is absorbed by the foam material and transformed into a gas for collection within exit path 190, passage through exit port 192 into the interior of handle assembly body 90. The gas exits through one or more of the exhaust ports 133, 135 in the handle assembly body.

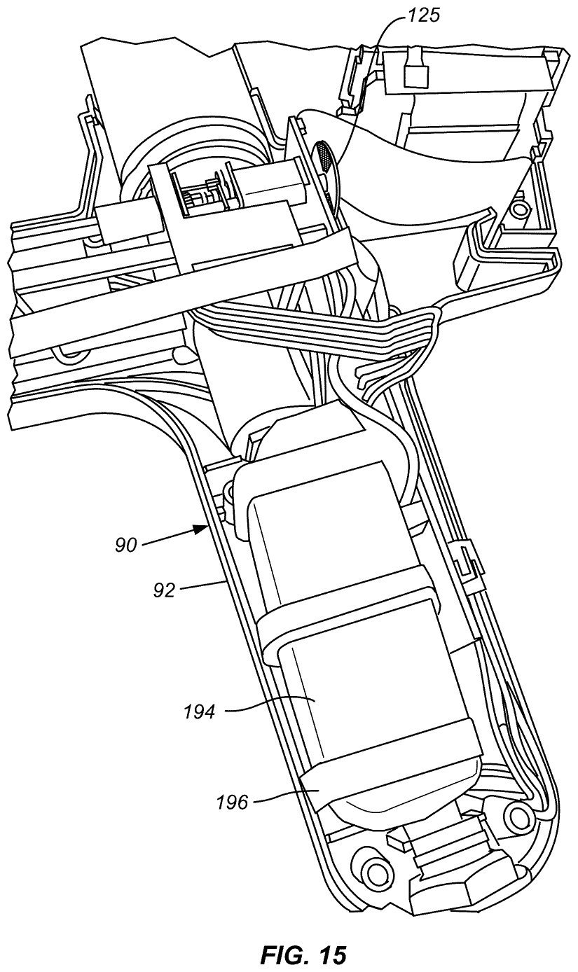

The transformation of the liquid refrigerant into a gas causes refrigerant venting chamber 180 to become quite cold. To prevent handgrip portion 92 of handle assembly body 90 from becoming too cold, refrigerant venting chamber 180 is wrapped with a thermal insulation material 194, see FIG. 15, and is spaced apart from the handle assembly body by insulating spacers 196. In this example 3 insulating spacers 196 are used to maintain a gap between refrigerant venting chamber 180 and handgrip portion 92 of handle assembly body 90. Thermal insulation material can be made of, for example, air, and insulating spacers 196 can be made of, for example, neoprene.

The pressure within the balloon 24 is communicated to a pressure transducer 143 in the controller connector 100. See FIGS. 9E and 9F. The pressure detecting lumen 32 of the catheter is fluidly coupled to the pressure transducer 143 in the controller through the pressure detecting passage 66 and the pressure detection port 68 of the connector body 23. The pressure detecting passage 66 and the pressure detecting port 68 are shown in FIG. 7B. The O-rings 88 on either side of the pressure detection port 68 form a seal within the controller connector 100, as shown in FIG. 9A. Pressure transducer 143 is coupled to controller 50 to provide a pressure signal thereto. For clarity connection wires from components to the controller 50 are omitted from the figures.

The controller 50 may be used to control the delivery of refrigerant and the rotation and translation of the delivery tube 30 and diffuser 36 within the balloon 24. The controller 50 includes circuitry connected to components including the foot pedal assembly 15, delivery solenoid 130, exhaust solenoid 136, linear drive motor 104, rotation motor 120, counter wheels 119, 125, pressure transducers 141, 143, heater 102, thermistor 137, optical sensor 165, current sensors, and accelerometer,

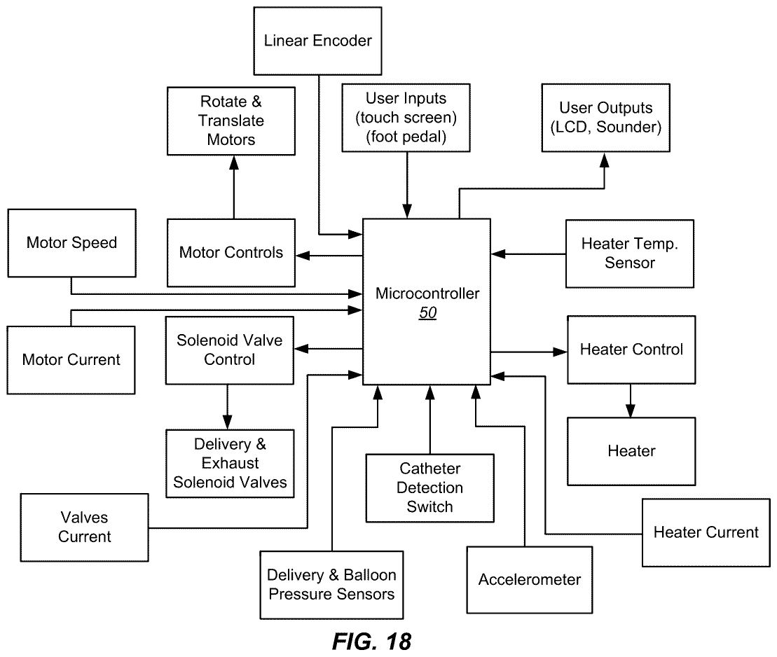

In embodiments, during release of refrigerant into the balloon 24 the controller 50 generates a pressure response curve from pressure data from the pressure transducer 141, which correlates to the inner diameter of the lumen to be treated. The controller 50 uses a pressure algorithm to determine the rate of speed for the linear actuator appropriate for treatment. In embodiments, a strain gauge or gauges on the balloon 24 may be used by the controller 50 to derive balloon diameter which corresponds to the inner diameter of the treated lumen. In embodiments the controller may be attached to forms of user interfaces in addition to or instead of those provided by foot pedal assembly 15 and LCD display 150, including buttons on the housing of the handle assembly and remote touch displays. FIG. 18 is a simplified diagram showing the basic organization of control electronics of the controller 50. An implementation is operative to enable the turbine to rotate from the flow rate provided by the controller, without specialized logic being made to the controller to support turbine operation. An advantage of these implementations is that there is compatibility between the turbine diffuser with earlier controllers operational with non-turbine diffusers. In embodiments, the control electronics may be connected to additional components including user outputs including lights and displays, additional temperature sensors, heater controllers, accelerometers, detector switches, and solenoid valves. The controller may contain treatment algorithms and the inputs of components may be used by the algorithms to adjust treatment parameters, for example duration, flow rate of refrigerant, translation distance and speed, and rotational angles and speed.

In embodiments, the catheter 12 may include an RFID tag or chip 198, see FIG. 9A, identifying properties of the catheter including size of balloon, angle of spray of diffuser. The controller 50 may receive this information from an RFID reader 200 in the handle assembly 14 and input the information into a treatment algorithm to adjust treatment parameters depending on the properties of the attached catheter. The RFID may be used for authentication purposes. For example, a non-conforming catheter (e.g. reused or overused catheter, or catheter made by an uncertified manufacturer) may be detected by the controller and the controller will lock out the device from operating with the non-conforming catheter attached. The RFID may further be used for orientation purposes to ensure catheter is oriented properly. The number of times or the length of use of a particular catheter 12 can also be monitored or controlled by providing unique identification information for each catheter to its associated RFID chip 198. It is preferred that connector body 23 be made of a material, such as polycarbonate, which facilitates the interrogation of the RFID chip 198 by the RFID reader 200. According to embodiments discussed herein, handle assembly 14 can be considered universal handle for use with a wide variety of catheters 12 because information regarding the catheter can be automatically provided to controller 50 within handle assembly 14 through the use of RFID chips 198 and RFID reader 200.

In embodiments, the user may select a treatment algorithm prior to initiating the treatment. Additionally, the user may be able to input various parameters to be used in the selected treatment algorithm. The information may include patient data, catheter information and number of treatments performed. The user interface for selecting and setting a treatment may the input on a separate device to permit programming remotely with reception by the controller wirelessly, wired or, for example, via a removable memory card.

The controller may record the number of uses of a catheter and save this information, or transmit this information to a central database to ensure no overuse of catheters. In embodiments, RFID tags on the catheter may be writeable so the controller can program catheter to be read in the future. The written material may include a lockout or a time of last use.

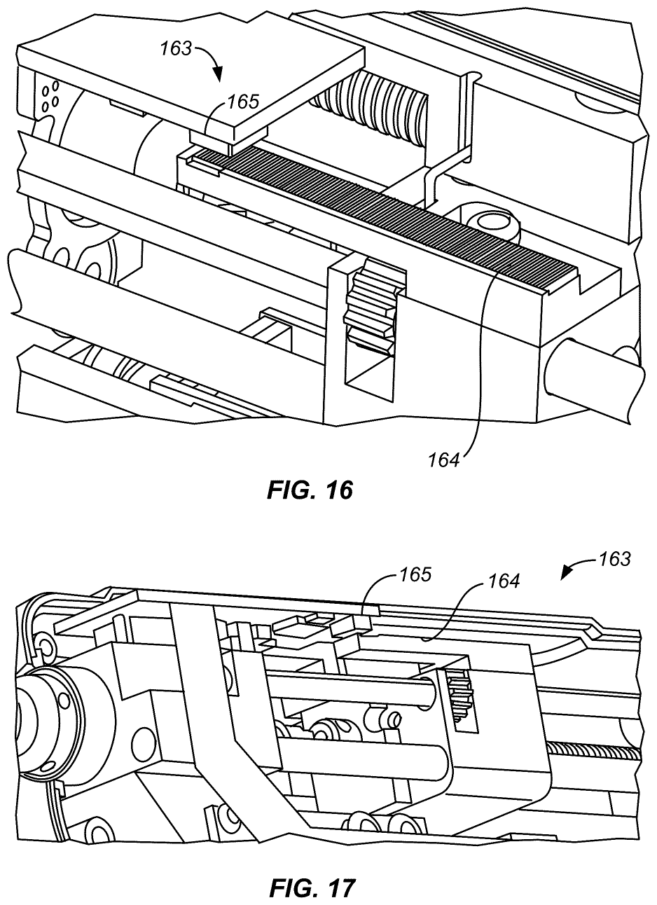

The following is an example of an ablation procedure. A cryogenic ablation catheter 12 is selected according to the treatment to be undertaken. An endoscope is inserted in the esophagus of a patient. Ablation catheter 12 with the plug 38 in the most distal position as shown in FIG. 2A is inserted into the proximal end 5 of the channel 8 of endoscopic tube 3. The plug 38 in the most distal position causes the diffuser 36 to push the flexible tip 48 away from the catheter shaft 16 causing the deflated balloon 24 to be in tension. The catheter 12 is inserted through the channel 8 until the balloon 24 exits the distal end 7. Using the monitor attached to the endoscope the user is able to see the balloon exit. With the system powered on, traveler 110 is translated by linear drive motor 104 to the connector load position of FIG. 9. A linear positioner 163 is used to ensure the proper linear position of traveler 110 relative to controller connector 100. Traveler 110 has a length of finely spaced lines 164, such as about 250 per inch, which are sensed by one or more optical sensors 165 carried by controller connector 100. See FIGS. 16 and 17.

The user selects, if desired, a treatment algorithm, inputs any necessary parameters, and taps on the refrigerant delivery foot pedal 132 to initially inflate the balloon 24. This initial inflation is required to visualize the location of target site relative to the lesion to be ablated. This initial inflation may include translating the diffuser to a position to allow for the balloon to be relaxed and no longer in tension. Instead of using a small amount of refrigerant to initially inflate the balloon 24, a syringe mounted to syringe coupler 56 could be used to initially inflate the balloon. An example of this position is shown in FIG. 2B. This can be followed by a short burst of refrigerant spray delivered onto inner surface of balloon 24 which inflates the balloon and allows the user to visually determine the location of the target site using the endoscope because of the freezing which occurs at tissue near the target site. If necessary, balloon 24 can be repositioned axially; this may or may not require the partial deflation balloon 24 followed by re-inflation of the balloon.

Once balloon 24 is properly positioned and inflated so that the nozzle ports 40 are directed at a portion of the lesion or other tissue to be cryogenically treated at the most distal end of the balloon, refrigerant is delivered to the diffuser to be sprayed on the interior wall of the balloon 24. While the refrigerant is being sprayed the diffuser can be translated toward the proximal end of the balloon or rotated about its axis using the pedal assembly 15. The flow rate of refrigerant, rotation rate and translation rate of the diffuser 36 are ideally set so that an ideal amount of refrigeration energy is received by each portion of the lesion to ensure ablation of the entire desired area. If the movement of the delivery tube assembly jams for any reason the controller will stop the delivery of refrigerant to prevent over ablation of tissue that may cause damage. Jams can be detected from the counter wheel 119 or monitoring current to the motors 104, 120.

Due to the direction of exhaust, it is beneficial to begin ablation from the distal end of the balloon as disclosed above because cool exhaust gas will pass over portions of the balloon interior surface that will subsequently be sprayed by refrigerant. This flow of exhaust gas therefore has a pre cooling effect which reduces the temperature prior to delivery which allows for less refrigerant to be used to achieve a desired ablation temperature. This pre-cooling effect is factored into the treatment algorithms.

The above descriptions may have used terms such as proximal, distal, above, below, top, bottom, over, under, et cetera. These terms may be used in the description and claims to aid understanding of the invention and not used in a limiting sense.

While implementations of the technology are disclosed by reference to the preferred embodiments and examples detailed above, it is to be understood that these examples are intended in an illustrative rather than in a limiting sense. It is contemplated that modifications and combinations will occur to those skilled in the art, which modifications and combinations will be within the spirit of the technology disclosed and the scope of the following claims. For example, in some situations it may be desired to rotate and translate simultaneously. In some examples such movement may be limited to preprogrammed movement rather than providing foot pedal assembly with such functionality.

One or more elements of one or more clauses/claims can be combined with elements of other clauses/claims.

### Clauses

The following clauses describe aspects of various examples of the technology described in this application.
01. A rotational turbine diffuser, having a distal end and a proximal end, and comprising:
   a non-rotatable discharge core 404 having an external portion 404a at the distal end and an internal portion 404b that is fluidly coupled to a delivery tube by a lumen 43 at the proximal end to (i) receive refrigerant and (ii) distribute refrigerant via internal pathways 41;
   a rotatable assembly 400 to receive the refrigerant and having a first plurality of cavities 49 for conducting the refrigerant to a plurality of orifices 40 in the rotatable assembly 400 to dispense the refrigerant, causing the rotatable assembly 400 to rotate about the internal portion 404b of the non-rotatable discharge core 404, thereby radially diffusing the refrigerant outward in a 360 degree discharge pattern; and
   a retainer (or means) 401 to secure the rotatable assembly 400 to the non-rotatable discharge core 404 and to permit the rotatable assembly 400 to rotate freely about the internal portion 404b.

02. A rotational turbine diffuser according to clause 01, wherein the retainer 401 includes a proximal retainer 401 fixedly mounted to the non-rotatable discharge core 404 on the proximal end.

03. A rotational turbine diffuser according to clause 01, wherein the rotatable assembly 400 comprises:
a rotatable discharge disk 403 having on a first side, the first plurality of cavities 49 to receive the refrigerant and to conduct the refrigerant to the plurality of orifices 40; and
a rotatable discharge cover 402 secured to the rotatable discharge disk 403, covering the first plurality of cavities 49, thereby forming viaways for the refrigerant

04. The rotational turbine diffuser according to clause 03,
wherein the first plurality of cavities 49 is located at a proximal side of the rotatable discharge disk 403,
wherein the rotatable discharge disk 403 further comprises a second plurality of cavities 49 located at a distal side of the rotatable discharge disk 403 and corresponding orifices 40, and
wherein the rotational assembly 400 further includes a second discharge cover 405 fitted over internal portion 404b to form additional viaways of on the distal side of the rotatable discharge disk 403 to provide refrigerant to the corresponding orifices 40 on the distal side of rotatable disk 403.

05. The rotational turbine diffuser according to clause 01, wherein two orifices 40, of the first plurality of orifices 40, are spaced at approximately 180 degrees circumferentially about the rotatable assembly 400.

06. The rotational turbine diffuser according to clause 01, wherein three orifices 40, of the first plurality of orifices 40, are spaced at approximately 120 degrees apart circumferentially about the rotatable assembly 400.

07. The rotational turbine diffuser according to clause 01, wherein four orifices 40, of the first plurality of orifices 40, are spaced at approximately 90 degrees apart circumferentially about the rotatable assembly 400.

08. The rotational turbine diffuser according to clause 01, wherein, when the rotational turbine diffuser is disposed within a balloon having an inner surface defining a balloon interior:
the rotation of the rotatable discharge disk 403 about the internal portion 404b of the non-rotatable discharge core 404, radially diffuses the refrigerant outward onto the inner surface of the balloon in the 360 degree discharge pattern.

09. The rotational turbine diffuser according to clause 01, further comprising:
one or more tubes 460 secured within one or more cavities 49, of the plurality of cavities 49, of the rotatable discharge disk 403, thereby reducing a diameter of one or more orifices 40 of the first plurality of orifices 40.

010. A rotational turbine diffuser, having a distal end and a proximal end, and comprising:
a non-rotatable discharge core 404 having an external portion 404a at the distal end and an internal portion 404b that is fluidly coupled to a delivery tube by a lumen 43 at the proximal end to (i) receive refrigerant and (ii) distribute refrigerant via internal pathways 41;
a rotatable discharge disk 403 to receive the refrigerant and having a first plurality of cavities 49 on a first side and a discharge cover 402 covering the first plurality of cavities 49 forming viaways for conducting the refrigerant to a plurality of orifices 40 in the rotatable discharge disk 403 to dispense the refrigerant, causing at least the rotatable discharge disk 403 to rotate about the internal portion 404b of the non-rotatable discharge core 404, thereby radially diffusing the refrigerant outward in a 360 degree discharge pattern; and
a retainer 401 fixedly mounted to the non-rotatable discharge core 404, securing the rotatable assembly 400 to the non-rotatable discharge core 404 and permitting the rotatable assembly 400 to rotate freely about the internal portion 404b.

001. A rotational turbine diffuser, having a distal end and a proximal end, and comprising:
a non-rotating discharge core 404 having an external portion 404a at the distal end and an internal portion 404b that is fluidly coupled to a delivery tube by a lumen 43 at the proximal end to (i) receive refrigerant and to (ii) distribute refrigerant via internal pathways 41;
a rotatable discharge disk 403 having a plurality of cavities 49 on a first side for conducting refrigerant to a plurality of orifices 40 in the rotatable discharge disk 403, that dispense refrigerant radially outward in a stream, causing the rotatable discharge disk 403 to rotate about the internal portion 404b of the non-rotating discharge core 404, thereby distributing the refrigerant radially outward in a 360 degree discharge pattern,
a discharge cover 402 bonded to the rotatable discharge disk 403 and covering the cavities 49 forming viaways for the refrigerant, and
a retainer means 401 fixedly mounted to the non-rotating discharge core 404 securing the rotatable discharge disk 403 and discharge cover 402.

002. The rotational turbine diffuser according to clause 010 or 001,
wherein the first plurality of cavities 49 is located at a proximal side of the rotatable discharge disk 403,
wherein the rotatable discharge disk 403 further comprises a second plurality of cavities 49 located at a distal side of the rotatable discharge disk 403 and corresponding orifices 40, and
wherein the rotational assembly 400 further includes a second discharge cover 405 fitted over internal portion 404b to form additional viaways of on the distal side of the rotatable discharge disk 403 to provide refrigerant to the corresponding orifices 40 on the distal side of rotatable disk 403.

003. The rotational turbine diffuser according to clause 010 or 001, wherein two orifices 40 are spaced at 180 degrees apart about the rotating discharge disk 403.

004. The rotational turbine diffuser according to clause 010 or 001, wherein three orifices 40 are spaced at 120 degrees apart about the rotating discharge disk 403.

005. The rotational turbine diffuser according to clause 010 or 001, wherein four orifices 40 are spaced at 90 degrees apart about the rotating discharge disk 403.

006. The rotational turbine diffuser according to clause 010 or 001, wherein whenever the diffuser is disposed within a balloon, the balloon having an inner surface defining a balloon interior:
rotation of the rotating discharge disk 403 about the internal portion 404b of the non-rotating discharge core 404, broadcasts the refrigerant radially outward onto the inner surface of the balloon in a 360 degree discharge pattern.

007. The rotational turbine diffuser according to clause 010 or 001, further comprising:
one or more tubes secured within one or more cavities 49 of the rotating discharge disk 403, thereby reducing diameter of orifices 49 associated with any cavities 49 in which tubes are placed.

1. An ablation assembly comprising:
a controller assembly 13 comprising a handle assembly 14, a controller 50, and a user control assembly 15 coupled to the controller, the handle assembly comprising a first connector element 99;
a cryogenic ablation catheter 12 comprising:
   a catheter shaft 16 having proximal and distal ends and a catheter shaft lumen extending between the proximal and distal ends;
   a connector 22 at the proximal end of the catheter shaft selectively connected to the first connector element of the handle assembly, the connector comprising a connector body 23 and a second connector element 38, the first and second connector elements being mating connector elements;
   an expandable and collapsible balloon 24 mounted to the distal end of the catheter shaft, the balloon having an inner surface defining a balloon interior;
   a delivery tube assembly comprising:
      a delivery tube 30 housed within the catheter shaft for axial and rotational movement relative to the catheter shaft, the delivery tube having a proximal end connected to the second connector element; and
      a rotational turbine diffuser 36, within the balloon, the rotational turbine diffuser 36 comprising (i) a non-rotating discharge core 404 having an external portion 404a at the distal end and an internal portion 404b fluidly coupled to the delivery tube 30 by a lumen 43 at the proximal end to (i) receive refrigerant and to (ii) distribute refrigerant via internal pathways 41, a rotatable discharge disk 403 to receive the refrigerant and having a first plurality of cavities 49 on a first side and a discharge cover 402 covering the first plurality of cavities 49 forming viaways for conducting the refrigerant to a plurality of orifices 40 in the rotatable discharge disk 403 to dispense the refrigerant, causing at least the rotatable discharge disk 403 to rotate about the internal portion 404b of the non-rotatable discharge core 404, thereby radially diffusing the refrigerant outward in a 360 degree discharge pattern; and, a retainer 401 fixedly mounted to the discharge core on the proximal end securing the discharge disk and discharge cover; and
the handle assembly comprising:
   a handle assembly body 90;
   a controller connector 100 mounted to the handle assembly body and defining the first connector element, the connector body securable to the controller connector;
   a traveler 110 movably mounted to the handle assembly body for movement along an axis towards and away from the controller connector, the second connector element securable to the traveler for axial movement therewith;
   a refrigerant fluid source 96 selectively fluidly coupled to a delivery line 118 by the refrigerant controller 130, 128, the delivery line having a distal end connected to the traveler, whereby the refrigerant delivery source can be fluidly coupled to the delivery tube at the second connector element;
   a linear driver 104, 108, 106 operably coupled to the traveler for moving the traveler along the axis;
   a rotary motion driver 120, 124, 122 operably coupled to the second connector element for selective rotation of the second connector element and the proximal end of the delivery tube therewith about the axis;
the user control assembly operably coupled to the refrigerant controller, the linear driver and the rotary motion driver, the user control assembly comprising user inputs permitting the user to actuate the refrigerant controller, the linear driver and the rotary motion driver;
whereby the user can control the rotation and translation of the rotational turbine diffuser within the balloon to direct refrigerant outwardly in a desired pattern towards the inner surface of the balloon according to the size and location of the treatment site.

2. The assembly according to clause 1, wherein the user control assembly 15 comprises a foot pedal assembly 15 spaced apart from the handle assembly 14 and connected to the handle assembly by a line 17, the foot pedal assembly comprising foot actuated input devices.

3. The assembly according to clause 2, wherein the foot pedal assembly 15 comprises a left movement foot pedal 140, a right movement foot pedal 142 and movement mode button 144 by which the user can change the mode of operation of the left and right movement foot pedals to actuate either the linear driver or the rotary motion driver.

4. The assembly according to clause 3, wherein:
the cryogenic ablation catheter 12 comprises an exhaust lumen 28 between the catheter shaft 16 and the delivery tube 30, the exhaust lumen having a distal end opening into the interior of the balloon;
the foot pedal assembly 15 comprises:
   a refrigerant delivery foot pedal 132 by which a user can actuate the refrigerant controller to supply refrigerant to the balloon 24; and
   a deflation button 134;
the handle assembly comprises an exhaust solenoid 136 operably coupled to the deflation button to permit the user to selectively exhaust gas from the balloon interior.

5. The assembly according to any of clauses 1-4 wherein:
the traveler is positionable along the axis at a first, eject position, a second, load position, and at a range of third, operational positions, the first, eject position being closest to the controller connector, the second, load position being between the first, eject positions and the third, operational positions, and further comprising:
means for automatically securing the connector body 23 to the controller connector 100 and the second connector element 38 to the traveler 110 when the connector is inserted into the connector receptacle and the traveler is at the second, load position; and
means for automatically releasing the connector body from the controller connector and the second connector element from the traveler when the traveler is at the first, eject position to permit removal of the connector from the handle assembly body.

6. The assembly according to clause 5, wherein:
the automatically securing means comprises means for simultaneously automatically securing the connector body 23 to the controller connector 100 and the second connector element 38 to the traveler 110 when the connector is inserted into the connector receptacle and the traveler is at the second, load position; and
the automatically releasing means comprises means for automatically releasing the connector body from the controller connector as the traveler moves to the first, eject position, and thereafter releasing the second connector element from the traveler to permit removal of the connector from the handle assembly body.

7. The assembly according to any of clauses 1-6 wherein:
the refrigerant fluid source 96 comprises a removable and replaceable refrigerant cartridge having a refrigerant discharge portion/tip 178 through which refrigerant can pass to the delivery line 118 via the refrigerant controller 130, 128 when the refrigerant fluid source is at an operational position;
the handle assembly body 90 comprises a refrigerant venting chamber 180 and a pathway 182, 184, 186 fluidly connecting the interior of the refrigerant venting chamber to a region adjacent to the tip of the refrigerant cartridge when the refrigerant cartridge has been displaced from the operational position during removal of the refrigerant cartridge from the handle assembly body 90;

whereby residual liquid refrigerant from the refrigerant cartridge can flow into the refrigerant venting chamber for transformation into a refrigerant gas, the refrigerant venting chamber having an exit port 192 to permit the refrigerant gas to exit the refrigerant venting chamber.

8. The assembly according to clause 7, wherein the exit port 192 opens into the handle assembly body 90, the handle assembly body having a plurality of exhaust ports 133, 135 opening into the ambient atmosphere.

9. The assembly according to any of clauses 1-8, wherein the traveler 110 is slideably supported on at least one bearing shaft 112 for movement of the traveler along the axis.

10. The assembly according to any of clauses 1-4 and 7-9, wherein:
the traveler is positionable along the axis at a first, eject position, a second, load position, in the range of third, operational positions, the first, eject position being closest to the controller connector, the second, load position being between the first, eject positions and the third, operational positions, and further comprising:
the rotary motion driver comprises:
   a rotation motor 120 drivingly connected to a non-cylindrical rotation shaft 122;
   a drive gear 126 mounted to traveler 110 for axial movement with the traveler, the drive gear also slideably mounted to the rotation shaft, whereby rotation of the rotation shaft causes the drive gear to rotate while axial movement of the traveler causes the drive gear to slide along the rotation shaft; and
   gear teeth 85 formed on second connector element 38 and rotatably coupled to the drive gear when the traveler is in either the second, load position or the third, operational position, so that rotation of the drive gear causes the second connector element 38 to rotate; and
the linear driver comprises a linear drive motor 104 connected to a threaded shaft 106 by a threaded shaft coupler 108, the threaded shaft threadably engaging the traveler 110 so that rotation of the threaded shaft causes the traveler to move axially.

11. The assembly according to any of clauses 1-10, wherein:
the connector 22 comprises an RFID device 198 containing information relating to the cryogenic ablation catheter 12; and
the handle assembly comprises an RFID reader 200 used to obtain information from the RFID device.

12. The assembly according to clause 11, wherein the RFID reader 200 is mounted to the controller connector 100, the connector body 23 being made of PEEK to enhance the communication between the RFID reader and the RFID device 198.

13. The assembly according to any of clauses 1-12, wherein the controller assembly comprises a controller 50, and the user control assembly is operably coupled to the refrigerant controller, the linear driver, and the rotary motion driver through the controller.

14. The assembly according to clause 13, wherein:
a pressure detecting lumen 32 extends along the catheter shaft 16 fluidly coupling the balloon interior and the connector body 23; and
the controller 50 is configured to use input received from a pressure transducer 143 operably coupled to the pressure detecting lumen 32 through the connector body 23 to detect a pressure within the balloon 24.

15. The assembly according to clause 13, wherein the controller 50 is configured to monitor the pressure and temperature of the refrigerant fluid source 96, whereby the status of the refrigerant can be monitored.

16. The assembly according to any of clauses 1-15, wherein the first connector element comprises a connector receptacle and the second connector element comprises a plug.

17. An ablation assembly comprising:
a handle assembly 14; and
a catheter 12 comprising:
   a catheter shaft 16 having distal and proximal ends;
   a balloon 24 at the distal end of the catheter shaft;
   a connector 22 at the proximal end of the catheter shaft;
   a delivery tube 30 extending between the balloon and the proximal end of the catheter shaft; and
   the connector comprising a connector body 23 secured to the proximal end of the catheter shaft and a plug 38 secured to the delivery tube, the plug and delivery tube therewith movable axially and rotationally relative to the catheter shaft;
the handle comprising an open portion for receipt of the plug and at least a portion of the connector body; and
a connector locking assembly comprising:
   a connector body locking slot 74 formed in and circumscribing the connector body 23;
   a plug locking slot 84 formed in and circumscribing the plug 38;
   a connector body locking element 160 mounted to the handle and positioned to engage the connector body locking slot 74 when the connector 22 is in a load state;
   a plug locking element 152, 154 mounted to the handle and positioned to simultaneously engage the plug locking slot 84 when the connector is in the load state, thereby simultaneously automatically connecting the plug 38 and the connector body 23 to the handle to place the connector in a load state prior to use; and
   a connector body locking element 160 mounted to the handle and positioned to disengage from the body locking slot when the connector is being placed in an eject state, and a plug locking element 152, 154 mounted to the handle and positioned to disengage from the body locking slot when the connector is in the eject state, thereby automatically releasing the connector body 23 and thereafter the plug 38 from the handle to permit the connector to be removed from the handle.

18. The ablation assembly according to clause 17, wherein the connector body locking element comprises a first spring 160 and the plug locking element comprises a second spring 152, 154.

19. The ablation assembly according to clause 18, wherein:
the connector body 23 comprises a tapered surface 162 engageable by the first spring 160 when the connector is placed into the load state; and
the plug 38 comprises a tapered surface 156 engageable by the second spring 152, 154 when the connector is placed into the load state.

20. The ablation assembly according to either of clauses 18 or 19, wherein:
the connector locking assembly comprises a ramp 170 engageable by the first spring 160 when the connector is placed into the eject state; and
the connector locking assembly comprises a ramp 174 engageable by the second spring 152, 154 when the connector is placed into the load state.

21. An ablation assembly comprising:
a handle assembly 14; and
a catheter 12 comprising:
   a catheter shaft 16 having distal and proximal ends;
   a balloon 24 at the distal end of the catheter shaft;
   a connector 22 at the proximal end of the catheter shaft;
   a delivery tube 30 extending between the balloon and the proximal end of the catheter shaft; and
   the connector comprising a connector body 23 secured to the proximal end of the catheter shaft and a plug 38 secured to the delivery tube, the plug and delivery tube therewith movable axially and rotationally relative to the catheter shaft;
the handle comprising an open portion for receipt of the plug and at least a portion of the connector body; and
a connector locking assembly comprising:
   means for simultaneously automatically connecting the plug 38 (84, 152, 154, 156, 158) and the connector body 23 (160, 174, 162) to the handle to place the connector in a load state prior to use; and
   means for automatically releasing the connector body (74, 110, 160, 170) and thereafter the plug (84, 110, 152, 154, 174) from the handle to place the connector in an eject state to permit the connector to be removed from the handle.

22. A handle assembly 14 for use with a cryogenic ablation assembly 10 comprising:
a handle body 90 having an interior;
a refrigerant fluid source 96 within the interior comprising a removable and replaceable refrigerant cartridge 96 from which refrigerant can pass for use by the cryogenic ablation assembly 10 when the refrigerant fluid source is at an operational position, the refrigerant source comprising a refrigerant discharge portion 178;
the handle body 90 containing a refrigerant venting chamber 180 and a pathway 182, 184, 186 fluidly connecting the interior of the refrigerant venting chamber to a region adjacent to the refrigerant discharge portion of the refrigerant cartridge when the refrigerant cartridge has been displaced from the operational position during removal of the refrigerant cartridge from the handle body 90;
the handle body comprising an exhaust port 133, 135 opening into the ambient atmosphere;
whereby residual liquid refrigerant from the refrigerant cartridge can flow into the refrigerant venting chamber for transformation into a refrigerant gas, the refrigerant venting chamber having an exit port 192 to permit the refrigerant gas to exit the refrigerant venting chamber and into a region external of the refrigerant venting chamber 180 and within the handle body 90, for passage through the exhaust port to the ambient atmosphere.
23. The handle assembly according to clause 22, wherein:
the refrigerant venting chamber 180 is filled with a material with an entrance path 188 and an exit path 190 formed in the material, the entrance path connected to the pathway; and
the exit path 190 terminating at the exit port 192;
whereby under a reduced pressure within the refrigerant venting chamber 180, the liquid refrigerant is absorbed by the foam material and transformed into a gas for collection within the exit path 190, passage through exit port 192 into said region external of the refrigerant venting chamber 180 and within the handle body 90.
24. The handle assembly according to either of clauses 22 or 23, further comprising:
a thermal insulation material 194 between the refrigerant venting chamber 180 and the handle body 90; and
spacers 196 between the thermal insulation material 194 and the handle body 90.

25. Catheter identification structure for a cryogenic ablation assembly 10 of the type including a handle assembly 14 and a catheter assembly, the catheter 12 comprising a catheter shaft 16 having distal and proximal ends, a connector 22 at the proximal end of the catheter shaft, the handle assembly 14 comprising an open portion for receipt of at least a portion of the connector, the catheter identification structure comprising:
an RFID device 198 carried by the connector 22 containing information relating to the catheter 12; and
an RFID reader 200 carried by the handle assembly used to obtain information from the RFID device.

26. The structure according to clause 25, wherein the connector 22 comprises a connector body 23 made of PEEK to enhance the communication between the RFID reader 200 and the RFID device 198.

Any and all patents, patent applications and printed publications referred to above are incorporated by reference.

What is claimed is:

1. A rotational turbine diffuser, having a distal end and a proximal end, and comprising:
a non-rotatable discharge core having an external portion at the distal end and an internal portion that is fluidly coupled to a delivery tube by a lumen at the proximal end to (i) receive refrigerant and (ii) distribute the refrigerant via internal pathways;
a rotatable assembly to receive the refrigerant and having a first plurality of cavities for conducting the refrigerant to a plurality of orifices in the rotatable assembly to dispense the refrigerant, causing the rotatable assembly to rotate about the internal portion of the non-rotatable discharge core, thereby radially diffusing the refrigerant outward in a 360 degree discharge pattern; and
a retainer to secure the rotatable assembly to the non-rotatable discharge core and to permit the rotatable assembly to rotate freely about the internal portion.

2. A rotational turbine diffuser according to claim 1, wherein the retainer includes a proximal retainer fixedly mounted to the non-rotatable discharge core on the proximal end.

3. A rotational turbine diffuser according to claim 1, wherein the rotatable assembly comprises:
a rotatable discharge disk having on a first side, the first plurality of cavities to receive the refrigerant and to conduct the refrigerant to the plurality of orifices; and
a rotatable discharge cover secured to the rotatable discharge disk, covering the first plurality of cavities, thereby forming viaways for the refrigerant.

4. The rotational turbine diffuser according to claim 3,
wherein the first plurality of cavities is located at a proximal side of the rotatable discharge disk,
wherein the rotatable discharge disk further comprises a second plurality of cavities located at a distal side of the rotatable discharge disk and corresponding orifices, and
wherein the rotational assembly further includes a second discharge cover fitted over internal portion to form additional viaways of on the distal side of the rotatable discharge disk to provide refrigerant to the corresponding orifices on the distal side of rotatable disk.

5. The rotational turbine diffuser according to claim 1, wherein two orifices, of the plurality of orifices, are spaced at approximately 180 degrees circumferentially about the rotatable assembly.

6. The rotational turbine diffuser according to claim 1, wherein three orifices, of the plurality of orifices, are spaced at approximately 120 degrees apart circumferentially about the rotatable assembly.

7. The rotational turbine diffuser according to claim 1, wherein four orifices, of the plurality of orifices, are spaced at approximately 90 degrees apart circumferentially about the rotatable assembly.

8. The rotational turbine diffuser according to claim 1, wherein, when the rotational turbine diffuser is disposed within a balloon having an inner surface defining a balloon interior:
rotation of the rotatable assembly about the internal portion of the non-rotatable discharge core, radially diffuses the refrigerant outward onto the inner surface of the balloon in the 360 degree discharge pattern.

9. The rotational turbine diffuser according to claim 1, further comprising:
one or more tubes secured within one or more cavities, of the first plurality of cavities, of the rotatable assembly, thereby reducing a diameter of one or more orifices of the plurality of orifices.

10. A rotational turbine diffuser, having a distal end and a proximal end, and comprising:
a non-rotatable discharge core having an external portion at the distal end and an internal portion that is fluidly coupled to a delivery tube by a lumen at the proximal end to (i) receive refrigerant and (ii) distribute refrigerant via internal pathways;
a rotatable discharge disk to receive the refrigerant and having a first plurality of cavities on a first side and a discharge cover covering the first plurality of cavities forming viaways for conducting the refrigerant to a plurality of orifices in the rotatable discharge disk to dispense the refrigerant, causing at least the rotatable discharge disk to rotate about the internal portion of the non-rotatable discharge core, thereby radially diffusing the refrigerant outward in a 360 degree discharge pattern; and
a retainer fixedly mounted to the non-rotatable discharge core, securing the rotatable discharge disk to the non-rotatable discharge core and permitting the rotatable discharge disk to rotate freely about the internal portion.

11. An ablation assembly comprising:
a control assembly comprising a handle assembly, a controller, and a user control assembly coupled to the controller, the handle assembly comprising a first connector element;
a cryogenic ablation catheter comprising:
a catheter shaft having a proximal end and a distal end and a catheter shaft lumen extending therebetween;
a connector at the proximal end of the catheter shaft selectively connected to the first connector element of the handle assembly, the connector comprising a connector body and a second connector element, the first and second connector elements being mating connector elements;
a balloon, expandable and collapsible, mounted to the distal end of the catheter shaft, the balloon having an inner surface defining a balloon interior;
a delivery tube assembly comprising:
a delivery tube housed within the catheter shaft for axial and rotational movement relative to the catheter shaft, the delivery tube having a proximal end connected to the second connector element; and
a rotational turbine diffuser, within the balloon, and comprising a non-rotatable discharge core having an external portion at the distal end and an internal portion that is fluidly coupled to the delivery tube by a lumen at the proximal end to (i) receive refrigerant and (ii) distribute refrigerant via internal pathways, a rotatable assembly to receive the refrigerant and having a first plurality of cavities for conducting the refrigerant to a plurality of orifices in the rotatable assembly to dispense the refrigerant, causing the rotatable assembly to rotate about the internal portion of the non-rotatable discharge core, thereby radially diffusing the refrigerant outward in a 360 degree discharge pattern; and
a retainer to secure the rotatable assembly to the non-rotatable discharge core and to permit the rotatable assembly to rotate freely about the internal portion; and
the handle assembly comprising:
a handle assembly body comprising a connector receptacle for receipt of the connector;
a controller connector mounted to the handle assembly body and defining the first connector element, the connector body securable to the controller connector;
a traveler movably mounted to the handle assembly body for movement along an axis towards and away from the controller connector, the second connector element securable to the traveler for axial movement therewith;
a refrigerant fluid source selectively fluidly coupled to a delivery line by a refrigerant controller, the delivery line having a distal end connected to the traveler, whereby the refrigerant fluid source can be fluidly coupled to the delivery tube at the second connector element;
a linear driver operably coupled to the traveler for moving the traveler along the axis; and
the user control assembly operably coupled to the refrigerant controller and the linear driver, the user control assembly comprising user inputs permitting a user to actuate the refrigerant controller and the linear driver;
whereby a user can control translation of the rotational turbine diffuser within the balloon to direct refrigerant outwardly in a full 360 degree cylindrical pattern towards the inner surface of the balloon according to a size and location of a treatment site.

12. The ablation assembly according to claim 11, wherein the retainer includes a retainer fixedly mounted to the non-rotatable discharge core on the proximal end.

13. The ablation assembly according to claim 11, wherein the rotatable assembly comprises:
a rotatable discharge disk having on a first side, the first plurality of cavities to receive the refrigerant and to conduct the refrigerant to the plurality of orifices; and
a rotatable discharge cover secured to the rotatable discharge disk, covering the first plurality of cavities, thereby forming viaways for the refrigerant.

14. The ablation assembly according to claim 13,
wherein the first plurality of cavities is located at a proximal side of the rotatable discharge disk,
wherein the rotatable discharge disk further comprises a second plurality of cavities located at a distal side of the rotatable discharge disk and corresponding orifices, and
wherein the rotational assembly further includes a second discharge cover fitted over internal portion to form additional viaways of on the distal side of the rotatable discharge disk to provide refrigerant to the corresponding orifices on the distal side of rotatable disk.

15. The ablation assembly according to claim 11, wherein two orifices, of the plurality of orifices, are spaced at approximately 180 degrees circumferentially about the rotatable assembly.

16. The ablation assembly according to claim 11, wherein three orifices, of the plurality of orifices, are spaced at approximately 120 degrees apart circumferentially about the rotatable assembly.

17. The ablation assembly according to claim 11, wherein four orifices, of the plurality of orifices, are spaced at approximately 90 degrees apart circumferentially about the rotatable assembly.

18. The ablation assembly according to claim 11, wherein when the rotational turbine diffuser is disposed within a balloon having an inner surface defining a balloon interior:
rotation of the rotatable assembly about the internal portion of the non-rotating discharge core, radially diffuses the refrigerant outward onto the inner surface of the balloon in a 360 degree discharge pattern.

19. The ablation assembly according to claim 11, where the rotational turbine diffuser further comprises:
one or more tubes secured within one or more cavities, of the first plurality of cavities, of the rotatable assembly, thereby reducing a diameter of one or more orifices of the plurality of orifices.
